(19) United States
(12) Patent Application Publication
(10) Pub. No.: US 2016/0166648 A1
(43) Pub. Date: Jun. 16, 2016

(54) Treatment of Multiple Sclerosis With Combination of Laquinimod and Interferon-Beta

(71) Applicants: Yossi Gilgun, Bat Hefer (IL); Nora Tarcic, Modiin (IL)

(72) Inventors: Yossi Gilgun, Bat Hefer (IL); Nora Tarcic, Modiin (IL)

(73) Assignee: Teva Pharmaceutical Industries, Ltd., Petach-Tikva (IL)

(21) Appl. No.: 15/050,005

(22) Filed: Feb. 22, 2016

Related U.S. Application Data
(63) Continuation of application No. 14/527,199, filed on Oct. 29, 2014, now abandoned.
Application No. 14/527,199 is a division of application No. 13/560,872, filed on Jul. 27, 2012, now abandoned.
(60) Provisional application No. 61/512,817, filed on Jul. 28, 2011.

(51) Int. Cl. A61K 38/21; A61K 9/00; A61K 47/18; A61K 47/02; A61K 47/26; A61K 31/4704; A61K 45/06

(52) U.S. Cl. CPC A61K 38/215; A61K 31/4704; A61K 9/0019; A61K 45/06; A61K 47/02; A61K 47/26; A61K 47/18

(57) ABSTRACT

This invention provides a method of treating a patient afflicted with multiple sclerosis or presenting a clinically isolated syndrome comprising administering to the patient laquinimod as an add-on therapy to or in combination with interferon-β. This invention also provides a package and a pharmaceutical composition comprising laquinimod and interferon-β for treating a patient afflicted with multiple sclerosis or presenting a clinically isolated syndrome. This invention also provides laquinimod for use as an add-on therapy or in combination with interferon-β in treating a patient afflicted with multiple sclerosis or presenting a clinically isolated syndrome. This invention further provides use of laquinimod and interferon-β in the preparation of a combination for treating a patient afflicted with multiple sclerosis or presenting a clinically isolated syndrome.

This application claims benefit of U.S. Provisional Application No. 61/512,817, filed Jul. 28, 2011, the entire content of which is hereby incorporated by reference herein.

Throughout this application, various publications are referred to by first author and year of publication. Full citations for these publications are presented in a References section immediately before the claims. Disclosures of the documents and publications cited are hereby incorporated by reference in their entireties into this application in order to more fully describe the state of the art as of the date of the invention described herein.

### BACKGROUND

Multiple Sclerosis (MS) is a neurological disease affecting more than 1 million people worldwide. It is the most common cause of neurological disability in young and middle-aged adults and has a major physical, psychological, social and financial impact on subjects and their families, friends and bodies responsible for health care (EMEA Guideline, 2006).

It is generally assumed that MS is mediated by some kind of autoimmune process possibly triggered by infection and superimposed upon a genetic predisposition. It is a chronic inflammatory condition that damages the myelin of the Central Nervous System (CNS). The pathogenesis of MS is characterized by the infiltration of autoreactive T-cells from the circulation directed against myelin antigens into the CNS (Bjartmar, 2002). In addition to the inflammatory phase in MS, axonal loss occurs early in the course of the disease and can be extensive over time, leading to the subsequent development of progressive, permanent, neurologic impairment and, frequently, severe disability (Neuhaus, 2003). Symptoms associated with the disease include fatigue, spasticity, ataxia, weakness, bladder and bowel disturbances, sexual dysfunction, pain, tremor, paroxysmal manifestations, visual impairment, psychological problems and cognitive dysfunction (EMEA Guideline, 2006). MS disease activity can be monitored by cranial scans, including magnetic resonance imaging (MRI) of the brain, accumulation of disability, as well as rate and severity of relapses. The diagnosis of clinically definite MS as determined by the Poser criteria (Poser, 1983) requires at least two neurological events suggesting demyelination in the CNS separated in time and in location. A clinically isolated syndrome (CIS) is a single monosymptomatic attack suggestive of MS, such as optic neuritis, brain stem symptoms, and partial myelitis. Patients with CIS that experience a second clinical attack are generally considered to have clinically definite multiple sclerosis (CDMS). Over 80 percent of patients with a CIS and MRI lesions go on to develop MS, while approximately 20 percent have a self-limited process (Brex, 2002; Frohman, 2003).

Various MS disease stages and/or types are described in Multiple Sclerosis Therapeutics (Duntiz, 1999). Among them, relapsing-remitting multiple sclerosis (RRMS) is the most common form at the time of initial diagnosis. Many subjects with RRMS have an initial relapsing-remitting course for 5-15 years, which then advances into the secondary progressive MS (SPMS) disease course. Relapses result from inflammation and demyelination, whereas restoration of nerve conduction and remission is accompanied by resolution of inflammation, redistribution of sodium channels on demyelinated axons and remyelination (Neuhaus, 2003; Noseworthy, 2000).

In April 2001, an international panel in association with the National MS Society of America recommended diagnostic criteria for multiple sclerosis. These criteria became known as the McDonald Criteria. The McDonald Criteria make use of MRI techniques and are intended to replace the Poser Criteria and the older Schumacher Criteria (McDonald, 2001). The McDonald Criteria was revised in March 2005 by an international panel (Polman, 2005) and updated again in 2010 (Polman, 2011).

Intervention with disease-modifying therapy at relapsing stages of MS is suggested to reduce and/or prevent accumulating neurodegeneration (Hohlfeld, 2000; De Stefano, 1999). There are currently a number of disease-modifying medications approved for use in relapsing MS (RMS), which includes RRMS and SPMS (The Disease Modifying Drug Brochure, 2006). These include interferon beta 1-a (Avonex® and Rebif®), interferon beta 1-b (Betaseron®), glatiramer acetate (Copaxone®), mitoxantrone (Novantrone®), natalizumab (Tysabri®) and fingolimod (Gilenya®). Most of them are believed to act as immunomodulators. Mitoxantrone and natalizumab are believed to act as immunesuppressants. However, the mechanisms of action of each have been only partly elucidated. Immunosuppressants or cytotoxic agents are used in some subjects after failure of conventional therapies. However, the relationship between changes of the immune response induced by these agents and the clinical efficacy in MS is far from settled (EMEA Guideline, 2006).

Other therapeutic approaches include symptomatic treatment which refers to all therapies applied to improve the symptoms caused by the disease (EMEA Guideline, 2006) and treatment of acute relapses with corticosteroids. While steroids do not affect the course of MS over time, they can reduce the duration and severity of attacks in some subjects.

Laquinimod

Laquinimod is a novel synthetic compound with high oral bioavailability which has been suggested as an oral formulation for the treatment of Multiple Sclerosis (MS) (Polman, 2005; Sandberg-Wollheim, 2005). Laquinimod and its sodium salt form are described, for example, in U.S. Pat. No. 6,077,851.

The mechanism of action of laquinimod is not fully understood. Animal studies show it causes a Th 1 (T helper 1 cell, produces pro-inflammatory cytokines) to Th2 (T helper 2 cell, produces anti-inflammatory cytokines) shift with an anti-inflammatory profile (Yang, 2004; Brück, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runstrom, 2006; Brück, 2011).

Laquinimod showed a favorable safety and tolerability profile in two phase III trials (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

Interferon beta (IFN-β)

Interferons (IFNs) are cytokines produced and released by host cells in response to the presence of pathogens and allow communication between cells to trigger the protective defenses of the immune system. IFN-β has been used over that past 15 years as treatment for RRMS. IFNs' complex mechanisms of action are not yet completely elucidated. Commercially available IFN-β include Avonex®, Betaseron®, Extavia® and Rebif®.

### Add-On/Combination Therapy

The effects of add-on or combination therapy using laquinimod and interferon-β on MS patients have not been reported.

The administration of two drugs to treat a given condition, such as multiple sclerosis, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of GA and interferon (IFN) has been experimentally shown to abrogate the clinical effectiveness of either therapy (Brod 2000). In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject. Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects (Vollmer, 2008; Rudick 2006; Kleinschmidt-DeMasters, 2005; Langer-Gould 2005).

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

Therefore, the state of the art at the time of filing is that the effects of an add-on or combination therapy of two drugs, in particular laquinimod and IFN-β, cannot be predicted until the results of a formal combination study are available.

### BRIEF DESCRIPTION OF THE DRAWINGS

FIG. 1: FIG. 1 is a graphical representation of the activity of interferon-β administered daily, subcutaneous (s.c). alone or in combination with laquinimod in chronic EAE in C57 BI mice. The graph shows the mean clinical score for the EAE rodents in each group (on the y-axis) against the days (on the x-axis).

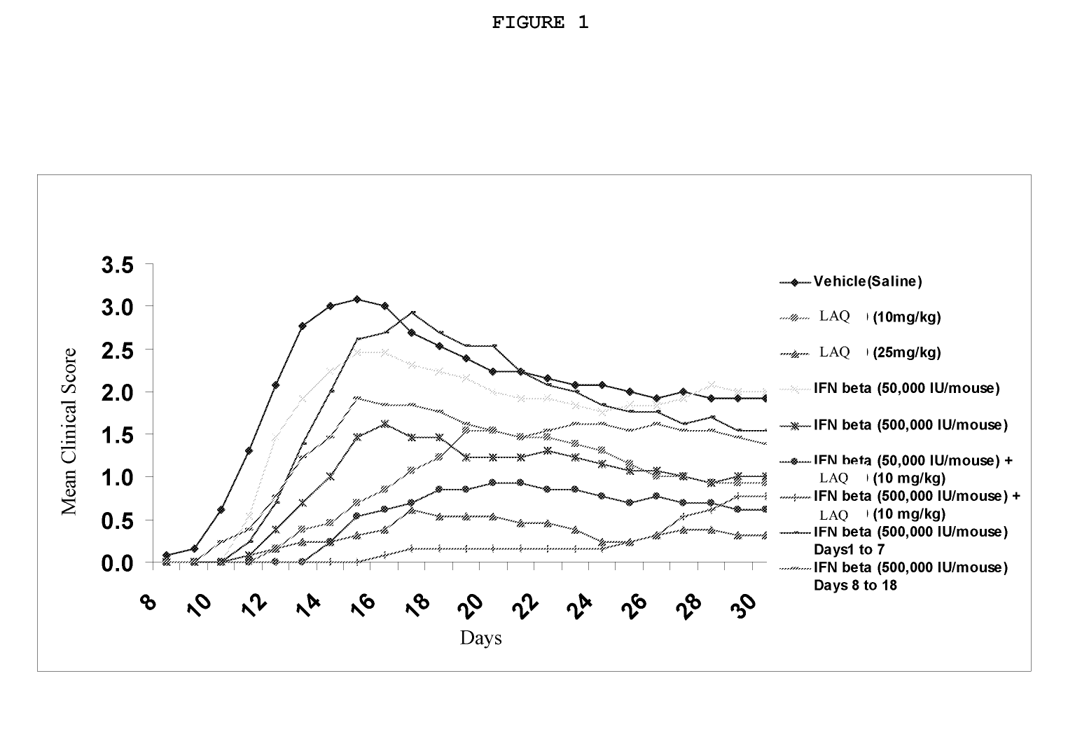

### SUMMARY OF THE INVENTION

This invention provides a method of treating a human patient afflicted with multiple sclerosis or presenting a clinically isolated syndrome comprising orally administering to the patient a daily dose of 0.6 mg laquinimod, and periodically administering to the patient a pharmaceutically effective amount of interferon-β, wherein the amounts when taken together is more effective to treat the human patient than when each agent is administered alone.

This invention provides a method of treating a human patient afflicted with multiple sclerosis or presenting a clinically isolated syndrome comprising periodically administering to the patient an amount of laquinimod and an amount of interferon-β, wherein the amounts when taken together are effective to treat the human patient.

This invention also provides a package comprising a) a first pharmaceutical composition comprising an amount of laquinimod and a pharmaceutically acceptable carrier; b) a second pharmaceutical composition comprising an amount of interferon-β and a pharmaceutically acceptable carrier; and c) instructions for use of the first and second pharmaceutical compositions together to treat a human patient afflicted with multiple sclerosis or presenting a clinically isolated syndrome.

This invention also provides laquinimod for use as an add-on therapy or in combination with interferon-β in treating a human patient afflicted with multiple sclerosis or presenting a clinically isolated syndrome.

This invention also provides a pharmaceutical composition comprising an amount of laquinimod and an amount of interferon-β for use in treating a human patient afflicted with multiple sclerosis or presenting a clinically isolated syndrome, wherein the laquinimod and the interferon-β are administered simultaneously or contemporaneously.

This invention also provides a pharmaceutical composition comprising an amount of laquinimod and an amount of interferon-β.

This invention also provides use of an amount of laquinimod and an amount of interferon-β in the preparation of a combination for treating a human patient afflicted with multiple sclerosis or presenting a clinically isolated syndrome wherein the laquinimod and the interferon-β are administered simultaneously or contemporaneously.

This invention also provides pharmaceutical composition comprising an amount of laquinimod for use in treating a subject afflicted with multiple sclerosis or presenting a clinically isolated syndrome as an add-on therapy or in combination with interferon-β by periodically administering the pharmaceutical composition and the interferon-β to the subject.

This invention further provides pharmaceutical composition comprising an amount of interferon-β for use treating a subject afflicted with multiple sclerosis or presenting a clinically isolated syndrome as an add-on therapy or in combination with laquinimod by periodically administering the pharmaceutical composition and the laquinimod to the subject.

### DETAILED DESCRIPTION OF THE INVENTION

This invention provides a method of treating a human patient afflicted with multiple sclerosis or presenting a clinically isolated syndrome comprising orally administering to the patient a daily dose of 0.6 mg laquinimod, and periodically administering to the patient a pharmaceutically effective amount of interferon-β, wherein the amounts when taken together is more effective to treat the human patient than when each agent is administered alone.

In one embodiment, the multiple sclerosis is relapsing multiple sclerosis. In another embodiment, the relapsing multiple sclerosis is relapsing-remitting multiple sclerosis.

In one embodiment, the amount of laquinimod and the amount of interferon-β when taken together is effective to reduce a symptom of multiple sclerosis in the human patient. In another embodiment, the symptom is a MRI-monitored multiple sclerosis disease activity, relapse rate, accumulation of physical disability, frequency of relapses, frequency of clinical exacerbation, brain atrophy, risk for confirmed progression, or time to confirmed disease progression.

In one embodiment, the accumulation of physical disability is assessed by the time to confirmed disease progression as measured by Kurtzke Expanded Disability Status Scale (EDSS) score. In another embodiment, the patient had an EDSS score of 0-5 prior to administration of laquinimod. In another embodiment, the patient had an EDSS score of 1-5.5 prior to administration of laquinimod. In another embodiment, the patient had an EDSS score of 0-5.5 prior to administration of laquinimod. In another embodiment, the patient had an EDSS score of 5.5 or greater prior to administration of laquinimod. In another embodiment, confirmed disease progression is a 1 point increase of the EDSS score. In yet another embodiment, confirmed disease progression is a 0.5 point increase of the EDSS score.

In one embodiment, time to confirmed disease progression is increased by 10-100%. In another embodiment, time to confirmed disease progression is increased by 20-80%. In another embodiment, time to confirmed disease progression is increased by 20-60%. In another embodiment, time to confirmed disease progression is increased by 30-50%. In yet another embodiment, time to confirmed disease progression is increased by at least 50%.

In one embodiment, laquinimod is laquinimod sodium. In another embodiment, the interferon-β is administered via subcutaneous injection or intramuscular injection.

In one embodiment, the interferon-β is interferon beta-1a. In another embodiment, the interferon-β is interferon beta-1b.

In one embodiment, the interferon-β is administered intramuscularly. In another embodiment, the interferon-β is administered subcutaneously. In another embodiment, the interferon-β is administered 1-5 times a month. In another embodiment, the interferon-β is administered 1-3 times a month. In another embodiment, the interferon-β is administered 1-5 times a week. In another embodiment, the interferon-β is administered 1-3 times a week. In another embodiment, the interferon-β is administered 1-5 times a day. In another embodiment, the interferon-β is administered 1-3 times a day. In another embodiment, the interferon-β is administered every other day. In yet another embodiment, the interferon-β is administered daily.

In one embodiment, the amount interferon-β administered is about 10-300 mcg. In another embodiment, the amount interferon-β administered is about 30-250 mcg. In another embodiment, the amount interferon-β administered is about 30-440 mcg. In another embodiment, the amount interferon-β administered is about 22-44 mcg. In another embodiment, the amount interferon-β administered is about 30 mcg. In another embodiment, the amount interferon-β administered is about 250 mcg.

In one embodiment, the interferon-β is interferon beta-1a and is administered intramuscularly at 30 mcg, once weekly. In another embodiment, the interferon-β is interferon beta-1b and is administered subcutaneously at 0.25 mg, every other day. In another embodiment, the interferon-β is interferon beta-lb and is administered subcutaneously at 0.25 mg, every other day. In yet another embodiment, the interferon-β is interferon beta-1a and is administered subcutaneously at 22-44 mcg, three times a week.

In one embodiment, the administration of laquinimod substantially precedes the administration of interferon-β. In another embodiment, the administration of interferon-β substantially precedes the administration of laquinimod.

In an embodiment, the human patient is receiving interferon-β therapy prior to initiating laquinimod therapy. In another embodiment, the human patient is receiving interferon-β therapy for at least 24 weeks prior to initiating laquinimod therapy. In another embodiment, the human patient is receiving interferon-β therapy for about 24 weeks prior to initiating laquinimod therapy. In another embodiment, the human patient is receiving interferon-β therapy for at least 28 weeks prior to initiating laquinimod therapy. In another embodiment, the human patient is receiving interferon-β therapy for about 28 weeks prior to initiating laquinimod therapy. In another embodiment, the human patient is receiving interferon-β therapy for at least 48 weeks prior to initiating laquinimod therapy. In another embodiment, the human patient is receiving interferon-β therapy for about 48 weeks prior to initiating laquinimod therapy. In another embodiment, the human patient is receiving interferon-β therapy for at least 52 weeks prior to initiating laquinimod therapy. In yet another embodiment, the human patient is receiving interferon-β therapy for about 52 weeks prior to initiating laquinimod therapy.

In one embodiment, the laquinimod is administered in the morning. In another embodiment, the laquinimod is administered at night. In one embodiment, the laquinimod is with food. In another embodiment, the laquinimod is administered without food.

In one embodiment, the interferon-β is administered in the morning. In another embodiment, the interferon-β is administered at night. In one embodiment, the interferon-β is administered with food. In another embodiment, the interferon-β is administered without food.

In one embodiment, the laquinimod is administered simultaneously with the interferon-β. In another embodiment, the laquinimod is administered contemporaneously with the interferon-β. In another embodiment, the laquinimod is administered immediately before or immediately after the interferon-β. In another embodiment, the laquinimod is administered within 1 hour before or after the interferon-β. In another embodiment, the laquinimod is administered within 3 hour before or after the interferon-β. In another embodiment, the laquinimod is administered within 6 hour before or after the interferon-β. In another embodiment, the laquinimod is administered within 12 hour before or after the interferon-β. In another embodiment, the laquinimod is administered within 24 hour before or after the interferon-β.

In one embodiment, the method further comprises administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

In an embodiment, the periodic administration of laquinimod and interferon-β continues for more than 30 days. In another embodiment, the periodic administration of laquinimod and interferon-β continues for more than 42 days. In yet another embodiment, the periodic administration of laquinimod and interferon-β continues for 6 months or more.

In one embodiment, the administration of laquinimod and interferon-β inhibits a symptom of relapsing multiple sclerosis by at least 20%. In another embodiment, the administration of laquinimod and interferon-β inhibits a symptom of relapsing multiple sclerosis by at least 30%. In another embodiment, the administration of laquinimod and interferon-β inhibits a symptom of relapsing multiple sclerosis by at least 40%. In another embodiment, the administration of laquinimod and interferon-β inhibits a symptom of relapsing multiple sclerosis by at least 50%. In another embodiment, the administration of laquinimod and interferon-β inhibits a symptom of relapsing multiple sclerosis by more than 100%. In another embodiment, the administration of laquinimod and interferon-β inhibits a symptom of relapsing multiple sclerosis by more than 300%. In yet another embodiment, the administration of laquinimod and interferon-β inhibits a symptom of relapsing multiple sclerosis by more than 1000%.

In one embodiment, each of the amount of laquinimod when taken alone, and the amount of interferon-β when taken alone is effective to treat the human patient. In another embodiment, either the amount of laquinimod when taken alone, the amount of interferon-β when taken alone, or each such amount when taken alone is not effective to treat the human patient.

This invention also provides a method of treating a human patient afflicted with multiple sclerosis or presenting a clinically isolated syndrome comprising periodically administering to the patient an amount of laquinimod and an amount of interferon-β (IFN-β), wherein the amounts when taken together are effective to treat the human patient. In one embodiment, the amount of laquinimod and the amount of IFN-β when taken together is more effective to treat the human patient than when each agent is administered alone.

In one embodiment, the multiple sclerosis is relapsing multiple sclerosis. In another embodiment, the relapsing multiple sclerosis is relapsing-remitting multiple sclerosis.

In one embodiment, the amount of laquinimod and the amount of interferon-β when taken together is effective to reduce a symptom of multiple sclerosis in the human patient. In another embodiment, the symptom is a MRI-monitored multiple sclerosis disease activity, relapse rate, accumulation of physical disability, frequency of relapses, decreased time to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work.

In one embodiment, the amount of laquinimod and the amount of interferon-β when taken together is effective to decrease or inhibit reduction of brain volume. In another embodiment, brain volume is measured by percent brain volume change (PBVC).

In one embodiment the amount of laquinimod and the amount of interferon-β when taken together is effective to increase time to confirmed disease progression. In another embodiment, time to confirmed disease progression is increased by 20-60%. In yet another embodiment, time to confirmed disease progression is increased by at least 50%.

In one embodiment, the amount of laquinimod and the amount of interferon-β when taken together is effective to decrease abnormalities observed in whole Brain MTR histogram.

In one embodiment, the accumulation of physical disability is measured by Kurtzke Expanded Disability Status Scale (EDSS) score. In another embodiment, the accumulation of physical disability is assessed by the time to confirmed disease progression as measured by Kurtzke Expanded Disability Status Scale (EDSS) score. In another embodiment, the patient had an EDSS score of 0-5.5 prior to administration of laquinimod. In another embodiment, the patient had an EDSS score of 1.5-4.5 prior to administration of laquinimod. In another embodiment, the patient had an EDSS score of 5.5 or greater prior to administration of laquinimod. In another embodiment, confirmed disease progression is a 1 point increase of the EDSS score. In yet another embodiment, confirmed disease progression is a 0.5 point increase of the EDSS score.

In one embodiment, impaired mobility is assessed by the Timed-25 Foot Walk test. In another embodiment, impaired mobility is assessed by the 12-Item Multiple Sclerosis Walking Scale (MSWS-12) self-report questionnaire In another embodiment, impaired mobility is assessed by the Ambulation Index (AI). In another embodiment, impaired mobility is assessed by the Six-Minute Walk (6MW) Test. In yet another embodiment, impaired mobility is assessed by the Lower Extremity Manual Muscle Test (LEMMT) Test.

In one embodiment, the amount of laquinimod and the amount of interferon-β when taken together is effective to reduce cognitive impairment. In another embodiment, cognitive impairment is assessed by the Symbol Digit Modalities Test (SDMT) score.

In one embodiment, general health status is assessed by the EuroQoL (EQ5D) questionnaire, Subject Global Impression (SGI) or Clinician Global Impression of Change (CGIC). In another embodiment, functional status is measured by the patient's Short-Form General Health survey (SF-36) Subject Reported Questionnaire score. In another embodiment, quality of life is assessed by SF-36, EQ5D, Subject Global Impression (SGI) or Clinician Global Impression of Change (CGIC). In another embodiment, the patient's SF-36 mental component summary score (MSC) is improved. In another embodiment, the patient's SF-36 physical component summary sore (PSC) is improved.

In one embodiment, fatigue is assessed by the EQ5D, the patient's Modified Fatigue Impact Scale (MFIS) score or the French valid versions of the Fatigue Impact Scale (EMIF-SEP) score. In another embodiment, symptom severity on work is measured by the work productivity and activities impairment General Health (WPAI-GH) questionnaire.

In one embodiment, laquinimod is laquinimod sodium. In another embodiment, laquinimod is administered via oral administration. In another embodiment, laquinimod is administered daily. In another embodiment, laquinimod is administered more often than once daily. In another embodiment, laquinimod is administered less often than once daily.

In one embodiment, the amount laquinimod administered is less than 0.6 mg/day. In another embodiment, the amount laquinimod administered is 0.1-40.0 mg/day. In another embodiment, the amount laquinimod administered is 0.1-2.5 mg/day. In another embodiment, the amount laquinimod administered is 0.25-2.0 mg/day. In another embodiment, the amount laquinimod administered is 0.5-1.2 mg/day. In another embodiment, the amount laquinimod administered is 0.25 mg/day. In another embodiment, the amount laquinimod administered is 0.3 mg/day. In another embodiment, the amount laquinimod administered is 0.5 mg/day. In another embodiment, the amount laquinimod administered is 0.6 mg/day. In another embodiment, the amount laquinimod administered is 1.0 mg/day. In another embodiment, the amount laquinimod administered is 1.2 mg/day. In another embodiment, n the amount laquinimod administered is 1.5 mg/day. In another embodiment, the amount laquinimod administered is 2.0 mg/day.

In one embodiment, a loading dose of an amount different form the intended dose is administered for a period of time at the start of the periodic administration. In another embodiment, the loading dose is double the amount of the intended dose. In yet another embodiment, the loading dose administered for two days at the start of the periodic administration.

In one embodiment, the interferon-β is administered via subcutaneous injection or intramuscular injection. In another embodiment, the interferon-β is interferon beta-1a and is administered intramuscularly at 30 mcg, once weekly. In another embodiment, the interferon-β is interferon beta-1b and is administered subcutaneously at 0.25 mg, every other day. In another embodiment, the interferon-β is interferon beta-1b and is administered subcutaneously at 0.25 mg, every other day. In another embodiment, the interferon-β is interferon beta-1a and is administered subcutaneously at 22-44 mcg, three times a week.

In one embodiment, the administration of laquinimod substantially precedes the administration of interferon-β . In another embodiment, the administration of interferon-β substantially precedes the administration of laquinimod. In another embodiment, the human patient is receiving interferon-β therapy prior to initiating laquinimod therapy. In another embodiment, the human patient is receiving interferon-β therapy for at least 24 weeks prior to initiating laquinimod therapy. In another embodiment, the human patient is receiving interferon-β therapy for at least 28 weeks prior to initiating laquinimod therapy. In another embodiment, the human patient is receiving interferon-β therapy for at least 48 weeks prior to initiating laquinimod therapy. In another embodiment, the human patient is receiving interferon-β therapy for at least 52 weeks prior to initiating laquinimod therapy.

In an embodiment, the method further comprises administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

In one embodiment, the periodic administration of laquinimod and interferon-β continues for at least 3 days. In another embodiment, the periodic administration of laquinimod and interferon-β continues for more than 30 days. In another embodiment, the periodic administration of laquinimod and interferon-β continues for more than 42 days. In another embodiment, the periodic administration of laquinimod and interferon-β continues for 8 weeks or more. In another embodiment, the periodic administration of laquinimod and interferon-β continues for at least 12 weeks. In another embodiment, the periodic administration of laquinimod and interferon-β continues for at least 24 weeks. In another embodiment, the periodic administration of laquinimod and interferon-β continues for more than 24 weeks. In another embodiment, the periodic administration of laquinimod and interferon-β continues for 6 months or more.

In an embodiment, the administration of laquinimod and interferon-β inhibits a symptom of relapsing multiple sclerosis by at least 20%. In another embodiment, the administration of laquinimod and interferon-β inhibits a symptom of relapsing multiple sclerosis by at least 30%. In another embodiment, the administration of laquinimod and interferon-β inhibits a symptom of relapsing multiple sclerosis by at least 50%. In another embodiment, the administration of laquinimod and interferon-β inhibits a symptom of relapsing multiple sclerosis by at least 70%. In another embodiment, the administration of laquinimod and interferon-β inhibits a symptom of relapsing multiple sclerosis by more than 100%. In another embodiment, the administration of laquinimod and interferon-β inhibits a symptom of relapsing multiple sclerosis by more than 300%. In yet another embodiment, the administration of laquinimod and interferon-β inhibits a symptom of relapsing multiple sclerosis by more than 1000%.

In one embodiment, each of the amount of laquinimod when taken alone, and the amount of interferon-β when taken alone is effective to treat the human patient. In another embodiment, either the amount of laquinimod when taken alone, the amount of interferon-β when taken alone, or each such amount when taken alone is not effective to treat the human patient.

This invention also provides a package comprising a) a first pharmaceutical composition comprising an amount of laquinimod and a pharmaceutically acceptable carrier; b) a second pharmaceutical composition comprising an amount of interferon-β and a pharmaceutically acceptable carrier; and c) instructions for use of the first and second pharmaceutical compositions together to treat a human patient afflicted with multiple sclerosis or presenting a clinically isolated syndrome.

In one embodiment, the first pharmaceutical composition is in liquid form. In another embodiment, the first pharmaceutical composition is in solid form. In another embodiment, the first pharmaceutical composition is in capsule form. In another embodiment, the first pharmaceutical composition is in tablet form. In another embodiment, the tablets are coated with a coating which inhibits oxygen from contacting the core. In another embodiment, the coating comprises a cellulosic polymer, a detackifier, a gloss enhancer, and pigment.

In one embodiment, the first pharmaceutical composition further comprises mannitol. In another embodiment, the first pharmaceutical composition further comprises an alkalinizing agent. In another embodiment, the alkalinizing agent is meglumine. In another embodiment, the first pharmaceutical composition further comprises an oxidation reducing agent.

In one embodiment, the first pharmaceutical composition is stable and free of an alkalinizing agent or an oxidation reducing agent. In another embodiment, the first pharmaceutical composition is free of an alkalinizing agent and free of an oxidation reducing agent.

In one embodiment, the first pharmaceutical composition is stable and free of disintegrant. In another embodiment, the first pharmaceutical composition further comprises a lubricant. In another embodiment, the lubricant is present in the composition as solid particles. In another embodiment, the lubricant is sodium stearyl fumarate or magnesium stearate.

In one embodiment the first pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In another embodiment, the filler is mannitol or lactose monohydrate.

In one embodiment, the package further comprises a desiccant. In another embodiment, the desiccant is silica gel.

In an embodiment, the first pharmaceutical composition is stable has a moisture content of no more than 4%. In another embodiment, laquinimod is present in the composition as solid particles.

In an embodiment, the package is a sealed packaging having a moisture permeability of not more than 15 mg/day per liter. In another embodiment, the sealed package is a blister pack in which the maximum moisture permeability is no more than 0.005 mg/day. In another embodiment, the sealed package is a bottle. In another embodiment, the bottle is closed with a heat induction liner. In another embodiment, the sealed package comprises an HDPE bottle. In another embodiment, the sealed package comprises an oxygen absorbing agent. In another embodiment, the oxygen absorbing agent is iron.

In an embodiment, the amount of laquinimod in the first composition is less than 0 6 mg. In another embodiment,the amount of laquinimod in the first composition is 0.1-40.0 mg. In another embodiment,the amount of laquinimod in the first composition is 0.1-2.5 mg. In another embodiment, the amount of laquinimod in the first composition is 0.25-2.0 mg. In another embodiment, the amount of laquinimod in the first composition is 0.5-1.2 mg. In another embodiment,the amount of laquinimod in the first composition is 0.25 mg. In another embodiment, the amount of laquinimod in the first composition is 0.3 mg. In another embodiment, the amount of laquinimod in the first composition is 0.5 mg. In another embodiment, the amount of laquinimod in the first composition is 0.6 mg. In another embodiment, the amount of laquinimod in the first composition is 1.0 mg. In another embodiment, the amount of laquinimod in the first composition is 1.2 mg. In another embodiment, the amount of laquinimod in the first composition is 1.5 mg. In yet another embodiment, the amount of laquinimod in the first composition is 2.0 mg.

This invention also provides laquinimod for use as an add-on therapy or in combination with interferon-β in treating a human patient afflicted with multiple sclerosis or presenting a clinically isolated syndrome.

This invention also provides a pharmaceutical composition comprising an amount of laquinimod and an amount of interferon-β for use in treating a human patient afflicted with multiple sclerosis or presenting a clinically isolated syndrome, wherein the laquinimod and the interferon-β are administered simultaneously or contemporaneously.

This invention also provides a pharmaceutical composition comprising an amount of laquinimod and an amount of interferon-β.

In one embodiment, the pharmaceutical composition is in liquid form. In another embodiment, the pharmaceutical composition is in solid form. In another embodiment, the pharmaceutical composition is in capsule form. In another embodiment, the pharmaceutical composition is in tablet form. In another embodiment, the tablets are coated with a coating which inhibits oxygen from contacting the core. In another embodiment, the coating comprises a cellulosic polymer, a detackifier, a gloss enhancer, and pigment.

In one embodiment, the pharmaceutical composition further comprises mannitol. In another embodiment, the pharmaceutical composition further comprises an alkalinizing agent. In another embodiment, the alkalinizing agent is meglumine. In yet another embodiment, the pharmaceutical composition further comprises an oxidation reducing agent.

In one embodiment, the pharmaceutical composition is free of an alkalinizing agent or an oxidation reducing agent. In another embodiment, the pharmaceutical composition is free of an alkalinizing agent and free of an oxidation reducing agent.

In one embodiment, the pharmaceutical composition is stable and free of disintegrant. In another embodiment, the pharmaceutical composition further comprises a lubricant. In another embodiment, the lubricant is present in the composition as solid particles. In another embodiment, the lubricant is sodium stearyl fumarate or magnesium stearate.

In an embodiment, the pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof In another embodiment, the filler is mannitol or lactose monohydrate.

In an embodiment, the amount of laquinimod in the composition is less than 0.6 mg. In another embodiment, the amount of laquinimod in the composition is 0.1-40.0 mg. In another embodiment, the amount of laquinimod in the composition is 0.1-2.5 mg. In another embodiment, the amount of laquinimod in the composition is 0.25-2.0 mg. In another embodiment, the amount of laquinimod in the composition is 0.5-1.2 mg. In another embodiment, the amount of laquinimod in the composition is 0.25 mg. In another embodiment, the amount of laquinimod in the composition is 0.3 mg. In another embodiment, the amount of laquinimod in the composition is 0.5 mg. In another embodiment, the amount of laquinimod in the composition is 0.6 mg. In another embodiment, the amount of laquinimod in the composition is 1.0 mg. In another embodiment, the amount of laquinimod in the composition is 1.2 mg. In another embodiment, the amount of laquinimod in the composition is 1.5 mg. In yet another embodiment, the amount of laquinimod in the composition is 2.0 mg.

This invention further provides use of an amount of laquinimod and an amount of interferon-β in the preparation of a combination for treating a human patient afflicted with multiple sclerosis or presenting a clinically isolated syndrome wherein the laquinimod and the interferon-β are administered simultaneously or contemporaneously.

This invention also provides pharmaceutical composition comprising an amount of laquinimod for use in treating a subject afflicted with multiple sclerosis or presenting a clinically isolated syndrome as an add-on therapy or in combination with interferon-β by periodically administering the pharmaceutical composition and the interferon-β to the subject.

This invention further provides pharmaceutical composition comprising an amount of interferon-β for use treating a subject afflicted with multiple sclerosis or presenting a clinically isolated syndrome as an add-on therapy or in combination with laquinimod by periodically administering the pharmaceutical composition and the laquinimod to the subject.

For the foregoing embodiments, each embodiment disclosed herein is contemplated as being applicable to each of the other disclosed embodiments. In addition, the elements recited in the packaging and pharmaceutical composition embodiments can be used in the method embodiments described herein.

### Laquinimod

Laquinimod mixtures, compositions, and the process for the manufacture thereof are described in, e.g., U.S. Pat. No. 6,077,851, U.S. Pat. No. 7,884,208, U.S. Pat. No. 7,989,473, U.S. Pat. No. 8,178,127, U.S. Application Publication No. 2010-0055072, U.S. Application Publication No. 2012-0010238, and U.S. Application Publication No. 2012-0010239, each of which is hereby incorporated by reference in its entireties into this application.

Use of laquinimod for treatment of various conditions, and the corresponding dosages and regimens, are described in U.S. Pat. No. 6,077,851 (multiple sclerosis, insulin-dependent diabetes mellitus, systemic lupus erythematosus, rheumatoid arthritis, inflammatory bowel disease, psoriasis, inflammatory respiratory disorder, atherosclerosis, stroke, and Alzhemier's disease), U.S. Application Publication No. 2011-0027219 (Crohn's disease), U.S. Application Publication No. 2010-0322900 (Relapsing-remitting multiple sclerosis), U.S. Application Publication No. 2011-0034508 (brain-derived neurotrophic factor (BDNF)-related diseases), U.S. Application Publication No. 2011-0218179 (active lupus nephritis), U.S. Application Publication No. 2011-0218203 (rheumatoid arthritis), U.S. Application Publication No. 2011-0217295 (active lupus arthritis), and U.S. Application Publication No. 2012-0142730 (reducing fatigue, improving quality of life, and providing neuroprotection in MS patients), each of which is hereby incorporated by reference in its entireties into this application.

### Commercially Available Interferon Beta (IFN-β)

Commercially available IFN-β include Avonex®, Betaseron®, Extavia® and Rebif®. The recommended Avonex® dose for treating MS is 30 mcg injected into a muscle once weekly. The recommended Betaseron® dose for treating MS is 0.25 mg injected (subcutaneously) every other day. The recommended Extavia® dose for treating MS is 0.25 mg, injected subcutaneously every other day. The recommended dose of Rebif® for treating MS is 22 mcg or 44 mcg, injected subcutaneously three times a week.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application. Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be formulated and made easy to swallow or chew; other solid forms include granules, and bulk powders.

Tablets may contain suitable binders, lubricants, disintegrating agents (disintegrants), coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-1actose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators (disintegrants) include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and 2007/146248.

General techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds).; Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is a method for treating a human patient afflicted with relapsing multiple sclerosis using laquinimod with interferon-β which provides a more efficacious treatment than each agent alone. The use of laquinimod for relapsing multiple sclerosis had been previously suggested in, e.g., U.S. Pat. No. 6,077,851. However, the inventors have surprisingly found that the combination of laquinimod and interferon-beta (IFN-β) is particularly effective for the treatment of relapsing multiple sclerosis as compared to each agent alone.

### Terms

As used herein, and unless stated otherwise, each of the following terms shall have the definition set forth below.

As used herein, “laquinimod” means laquinimod acid or a pharmaceutically acceptable salt thereof

As used herein, an “amount” or “dose” of laquinimod as measured in milligrams refers to the milligrams of laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion.

As used herein, “about” in the context of a numerical value or range means ±10% of the numerical value or range recited or claimed.

As used herein, a composition that is “free” of a chemical entity means that the composition contains, if at all, an amount of the chemical entity which cannot be avoided although the chemical entity is not part of the formulation and was not affirmatively added during any part of the manufacturing process.

For example, a composition which is “free” of an alkalizing agent means that the alkalizing agent, if present at all, is a minority component of the composition by weight. Preferably, when a composition is “free” of a component, the composition comprises less than 0.1 wt %, 0.05 wt %, 0.02 wt %, or 0.01 wt % of the component.

As used herein, “alkalizing agent” is used interchangeably with the term “alkaline-reacting component” or “alkaline agent” and refers to any pharmaceutically acceptable excipient which neutralizes protons in, and raises the pH of, the pharmaceutical composition in which it is used.

As used herein, “oxidation reducing agent” refers to a group of chemicals which includes an “antioxidant”, a “reduction agent” and a “chelating agent”.

As used herein, “antioxidant” refers to a compound selected from the group consisting of tocopherol, methionine, glutathione, tocotrienol, dimethyl glycine, betaine, butylated hydroxyanisole, butylated hydroxytoluene, turmerin, vitamin E, ascorbyl palmitate, tocopherol, deteroxime mesylate, methyl paraben, ethyl paraben, butylated hydroxyanisole, butylated hydroxytoluene, propyl gallate, sodium or potassium metabisulfite, sodium or potassium sulfite, alpha tocopherol or derivatives thereof, sodium ascorbate, disodium edentate, BHA (butylated hydroxyanisole), a pharmaceutically acceptable salt or ester of the mentioned compounds, and mixtures thereof.

The term “antioxidant” as used herein also refers to Flavonoids such as those selected from the group of quercetin, morin, naringenin and hesperetin, taxifolin, afzelin, quercitrin, myricitrin, genistein, apigenin and biochanin A, flavone, flavopiridol, isoflavonoids such as the soy isoflavonoid, genistein, catechins such as the tea catechin epigallocatechin gallate, flavonol, epicatechin, hesperetin, chrysin, diosmin, hesperidin, luteolin, and rutin.

As used herein, “reduction agent” refers to a compound selected from the group consisting of thiol-containing compound, thioglycerol, mercaptoethanol, thioglycol, thiodiglycol, cysteine, thioglucose, dithiothreitol (DTT), dithio-bis-maleimidoethane (DTME), 2,6-di-tert-butyl-4-methylphenol (BHT), sodium dithionite, sodium bisulphite, formamidine sodium metabisulphite, and ammonium bisulphite.”

As used herein, “chelating agent” refers to a compound selected from the group consisting of penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine, triethylenetetraamine and tris(2-carboxyethyl) phosphine (TCEP), ferrioxamine, CP94, EDTA, deferoxainine B (DFO) as the methanesulfonate salt (also known as desferrioxanilne B mesylate (DFOM)), desferal from Novartis (previously Ciba-Giegy), and apoferritin.

As used herein, a pharmaceutical composition is “stable” when the composition preserves the physical stability/integrity and/or chemical stability/integrity of the active pharmaceutical ingredient during storage. Furthermore, “stable pharmaceutical composition” is characterized by its level of degradation products not exceeding 5% at 40° C./75% RH after 6 months or 3% at 55° C./75% RH after two weeks, compared to their level in time zero.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the laquinimod and the IFN-β. In this case, the combination may be the admixture or separate containers of the laquinimod and the IFN-β that are combined just prior to administration. Contemporaneous administration refers to the separate administration of the laquinimod and the IFN-β at the same time, or at times sufficiently close together that a synergistic activity relative to the activity of either the laquinimod or the IFN-β alone is observed.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding laquinimod therapy to a patient already receiving IFN-β therapy.

As used herein, “effective” when referring to an amount of laquinimod and/or interferon-beta (IFN-β) refers to the quantity of laquinimod and/or interferon-beta (IFN-β) that is sufficient to yield a desired therapeutic response without undue adverse side effects (such as toxicity, irritation, or allergic response) commensurate with a reasonable benefit/risk ratio when used in the manner of this invention.

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition.

“Treating” as used herein encompasses, e.g., inducing inhibition, regression, or stasis of a disease or disorder, e.g., RMS, or lessening, suppressing, inhibiting, reducing the severity of, eliminating or substantially eliminating, or ameliorating a symptom of the disease or disorder. “Treating” as applied to patients presenting CIS can mean delaying the onset of clinically definite multiple sclerosis (CDMS), delaying the progression to CDMS, reducing the risk of conversion to CDMS, or reducing the frequency of relapse in a patient who experienced a first clinical episode consistent with multiple sclerosis and who has a high risk of developing CDMS.

“Inhibition” of disease progression or disease complication in a subject means preventing or reducing the disease progression and/or disease complication in the subject.

A “symptom” associated with RMS includes any clinical or laboratory manifestation associated with RMS and is not limited to what the subject can feel or observe.

As used herein, “a subject afflicted with relapsing multiple sclerosis” means a subject who has been clinically diagnosed to have relapsing multiple sclerosis (RMS) which includes relapsing-remitting multiple sclerosis (RRMS) and Secondary Progressive multiple sclerosis (SPMS).

As used herein, a subject at “baseline” is as subject prior to administration of laquinimod.

A “patient at risk of developing MS” (i.e. clinically definite MS) as used herein is a patient presenting any of the known risk factors for MS. The known risk factors for MS include any one of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB1, IL7R-alpha and IL2R-alpha), and immunological components (viral infection such as by Epstein-Barr virus, high avidity CD4³⁰ T cells, CD8⁺ T cells, anti-NF-L, anti-CSF 114(Glc)).

“Clinically isolated syndrome (CIS)” as used herein refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter.

“Relapse Rate” is the number of confirmed relapses per unit time. “Annualized relapse rate” is the mean value of the number of confirmed relapses of each patient multiplied by 365 and divided by the number of days that patient is on the study drug.

“Expanded Disability Status Scale” or “EDSS” is a rating system that is frequently used for classifying and standardizing the condition of people with multiple sclerosis. The score ranges from 0.0 representing a normal neurological exam to 10.0 representing death due to MS. The score is based upon neurological testing and examination of functional systems (FS), which are areas of the central nervous system which control bodily functions. The functional systems are: Pyramidal (ability to walk), Cerebellar (coordination), Brain stem (speech and swallowing), Sensory (touch and pain), Bowel and bladder functions, Visual, Mental, and Other (includes any other neurological findings due to MS) (Kurtzke J F, 1983).

A “confirmed progression” of EDSS, or “confirmed disease progression” as measured by EDSS score is defined as a 1 point increase from baseline EDSS sustained for at least 3 months. In addition, confirmation of progression cannot be made during a relapse.

“Adverse event” or “AE” means any untoward medical occurrence in a clinical trial subject administered a medicinal product and which does not have a causal relationship with the treatment. An adverse event can therefore be any unfavorable and unintended sign including an abnormal laboratory finding, symptom, or diseases temporally associated with the use of an investigational medicinal product, whether or not considered related to the investigational medicinal product.

“Gd-enhancing lesion” refers to lesions that result from a breakdown of the blood-brain barrier, which appear in contrast studies using gandolinium contrast agents. Gandolinium enhancement provides information as to the age of a lesion, as Gd-enhancing lesions typically occur within a six week period of lesion formation.

“Magnetization Transfer Imaging” or “MTI” is based on the magnetization interaction (through dipolar and/or chemical exchange) between bulk water protons and macromolecular protons. By applying an off resonance radio frequency pulse to the macromolecular protons, the saturation of these protons is then transferred to the bulk water protons. The result is a decrease in signal (the net magnetization of visible protons is reduced), depending on the magnitude of MT between tissue macromolecules and bulk water. “MT” or “Magnetization Transfer” refers to the transfer of longitudinal magnetization from the hydrogen nuclei of water that have restricted motion to the hydrogen nuclei of water that moves with many degrees of freedom. With MTI, the presence or absence of macromolecules (e.g. in membranes or brain tissue) can be seen (Mehta, 1996; Grossman, 1994).

“Magnetization Resonance Spectroscopy” or “MRS” is a specialized technique associated with magnetic resonance imaging (MRI). MRS is used to measure the levels of different metabolites in body tissues. The MR signal produces a spectrum of resonances that correspond to different molecular arrangements of the isotope being “excited”. This signature is used to diagnose certain metabolic disorders, especially those affecting the brain, (Rosen, 2007) as well as to provide information on tumor metabolism (Golder, 2007).

As used herein “mobility” refers to any ability relating to walking, walking speed, gait, strength of leg muscles, leg function and the ability to move with or without assistance. Mobility can be evaluated by one or more of several tests including but not limited to Ambulation Index, Time 25 foot walk, Six-Minute Walk (6MW), Lower Extremity Manual Muscle Test (LEMMT) and EDSS. Mobility can also be reported by the subject, for example by questionnaires, including but not limited to 12-Item Multiple Sclerosis Walking Scale (MSWS-12). Impaired Mobility refers to any impairment, difficulty or disability relating to mobility.

“T1-weighted MRI image” refers to an MR-image that emphasizes T1 contrast by which lesions may be visualized. Abnormal areas in a T1-weighted MRI image are “hypointense” and appear as dark spots. These spots are generally older lesions.

“T2-weighted MRI image” refers to an MR-image that emphasizes T2 contrast by which lesions may be visualized. T2 lesions represent new inflammatory activity.

The “Six-Minute Walk (6MW) Test” is a commonly used test developed to assess exercise capacity in patients with COPD (Guyatt, 1985). It has been used also to measure mobility in multiple sclerosis patients (Clinical Trials Website).

The “Timed-25 Foot Walk” or “T25-FW” is a quantitative mobility and leg function performance test based on a timed 25-walk. The patient is directed to one end of a clearly marked 25-foot course and is instructed to walk 25 feet as quickly as possible, but safely. The time is calculated from the initiation of the instruction to start and ends when the patient has reached the 25-foot mark. The task is immediately administered again by having the patient walk back the same distance. Patients may use assistive devices when doing this task. The score for the T25-FW is the average of the two completed trials. This score can be used individually or used as part of the MSFC composite score (National MS Society Website).

One of the central symptoms of multiple sclerosis is fatigue. Fatigue can be measured by several tests including but not limited to decrease of French valid versions of the Fatigue Impact Scale (EMIF-SEP) score, and European Quality of Life (EuroQoL) Questionnaire (EQSD). Other tests, including but not limited to Clinician Global Impression of Change (CGIC) and Subject Global Impression (SGI), as well as EQ-5D, can be used to evaluate the general health status and quality of life of MS patients.

“Ambulation Index” or “AI” is a rating scale developed by Hauser et al. to assess mobility by evaluating the time and degree of assistance required to walk 25 feet. Scores range from 0 (asymptomatic and fully active) to 10 (bedridden). The patient is asked to walk a marked 25-foot course as quickly and safely as possible. The examiner records the time and type of assistance (e.g., cane, walker, crutches) needed. (Hauser, 1983)

“EQ-5D” is a standardized questionnaire instrument for use as a measure of health outcome applicable to a range of health conditions and treatments. It provides a simple descriptive profile and a single index value for health status that can be used in the clinical and economic evaluation of health care as well as population health surveys. EQ-5D was developed by the “EuroQoL” Group which comprises a network of international, multilingual, multidisciplinary researchers, originally from seven centers in England, Finland, the Netherlands, Norway and Sweden. The EQ-5D questionnaire is in the public domain and can be obtained from EuroQoL.

“SF-36” is a multi-purpose, short-form health survey with 36 questions which yields an 8-scale profile of functional health and well-being scores as well as psychometrically-based physical and mental health summary measures and a preference-based health utility index. It is a generic measure, as opposed to one that targets a specific age, disease, or treatment group. The survey is developed by and can be obtained from QualityMetric, Inc. of Providence, R.I.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5mg/day” includes 0.1 mg/day, 0.2 mg/day, 0.3 mg/day, etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Assessment Of Add-on Effect Of Laquinimod In Mice Treated With Glatiramer Acetate (GA) Or Interferon-beta (IFN-β)

Mice were treated with a sub-optimal dose of Laquinimod (10mg/kg) alone or add on glatiramer acetate (12.5 mg/kg) or IFN-β (500,000 IU/mouse). In both cases, the combined treatment resulted in improved efficacy when compared to each treatment alone.

### Example 2

### Activity of Interferon-β Administered Daily, Subcutaneous (s.c). Alone or in Combination with Laquinimod in Chronic EAE in C57 BI Mice

Experimental autoimmune encephalomyelitis (EAE) is an animal model (mostly used with rodents) of the human CNS demyelinating diseases, including MS. MOG induced EAE in the C57B1 strain of mice was selected, as it is an established EAE model to test the efficacy of candidate molecule for MS treatement.

In this experiment interferon-β is administered daily, subcutaneous (s.c). alone or in combination with laquinimod to chronic MOG induced EAE in C57 BI mice. Both were administered from the beginning of the study in the MOG induced EAE in C57B1 mice.

General Design

Disease was induced in all mice by the injection of the encephalitogenic emulsion (MOG/CFA) and intraperitoneal injection of Pertussis toxin on the first day and 48 hours later. IFN-β at dose levels of 50,000 and 5000,000 IU/mouse was administered by the subcutaneous route, once daily (QD). Laquinimod at dose levels of 10 and 25 mg/mouse was administered by the oral route, once daily (QD). Both IFN-β and laquinimod were administered prophylactic from disease induction—Day 1 until termination of the study. Two additional groups of IFN-β at dose level of 500,000 were treated either prophylactic (Day 1-7) or from onset (Day 8-18) to study activity of IFN-β in prophylactic and therapeutic regime.

Materials

Interferon beta-1a (IFN-β ) (Rebif®, 44 μg/0.5 ml/syringe, equivalent to 1.2×10⁷ units (IU)/0.5 ml/syringe), Laquinimod, PBS (Sigma), Pertussiis toxin (Sigma), MOG 35-55 (Mnf Novatide), Complete Freund's Adjuvant (CFA) (Signma), Saline (Mnf-DEMO S.A).

Healthy, nulliparous, non-pregnant female mice of the C57BL/6 strain were used in the study. The animals weighed 18-22 grams and were approximately 8 weeks old on receipt. The body weights of the animals were recorded on the day of delivery. Overtly healthy animals were assigned to study groups arbitrarily before treatment commenced.

Procedures

EAE was induced by injecting the ecephalitogenic mixture (emulsion) consisting of MOG (150.0 μg/mouse) and CFA containing M. tuberculosis (1mg MG/ml CFA). A volume of 0.2 ml of encephalitogenic emulsion was injected subcutaneously into the flanks of each mouse (Dose=0.15 mg MOG and 0.2 mg MT/mouse) Pertussis toxin in 0.2 ml dosage volume is injected intraperitonieally on the day of induction and 48 hours later (total amount is 0.2 μg/mouse; 100.0 ng/0.2 ml/mouse).

The mice were allocated to the following treatment groups of 13 mice each.

TABLE 1
Experimental design

| | Treatment | | Admin- | |
|---|---|---|---|---|
| | Groups (treat- | | istration | |
| Group | ment initiation) | dose/day | Route | Regimen |
| 1 | Vehicle | 10 ml/kg | SC, QD | LAQUINIMOD |
| 2 | LAQUINIMOD | 10 mg/kg | Oral, QD | from Day 1 to |
| 2 | LAQUINIMOD | 25 mg/kg | Oral, QD | 30 daily & |
| 4 | IFN-β | 50,000 | SC, QD | IFN-β from |
| | | IU/mouse | | Day 1 to 18 |
| 5 | IFN-β | 500,000 | SC, QD | daily |
| | | IU/mouse | | |
| 6 | LAQUINIMOD + | 10 mg/kg + | Oral | |
| | IFN-β | 50,000 | (QD) + | |
| | | IU/mouse | SC, (QD) | |
| 7 | LAQUINIMOD + | 10 mg/kg + | Oral | |
| | IFN-β | 500,000 | (QD) + | |
| | | IU/mouse | SC (QD) | |
| 8 | IFN-β | 500,000 | SC, QD | Days 1 to 7 |
| | | IU/mouse | | (prophylactic) |
| 9 | IFN-β | 500,000 | | Days 8 to 18 |
| | | IU/mouse | | (therapeutic) |

The mice were administered with the various concentrations of IFN-β (2.5×10⁶ and 2.5×10⁵ IU/ml) at volume dose level of 200 μl/mouse by subcutaneous route equivalent to 50,000 and 500,000 IU/mouse respectively.

The laquinimod formulation was administered from Day 1, once daily (QD). Four hours interval was maintained between administration of laquinimod and IFN-β.

Experimental Observations

All animals were examined once daily to detect if any are moribund. Mice were also weighed once weekly. Further, the mice were observed daily from the 8^{th} day post-EAE induction and EAE clinical signs were scored. The scores were recorded on observation cards according to the grades described in Table 2 below.

TABLE 2
Evaluation of the EAE clinical signs

| Score | Signs | Description |
|---|---|---|
| 0 | Normal behavior | No neurological signs. |
| 1 | Limp tail | Part or the whole tail is limp and droopy. |
| 2 | righting reflex | Animal has difficulties rolling onto his feet |
| | | when laid on its back |
| 3 | Hind leg weakness | wobbly walk - when the mouse walks the |
| | | hind legs are unsteady |
| 4 | Hind leg paralysis | The mouse drags its hind legs but is able to |
| | | move around using its fore legs |
| 5 | Full paralysis | The mouse can't move around, it looks |
| | | thinner and emaciated. |
| 6 | Moribund/Death | |

All mice with score 1 and above were considered sick. When the first clinical sign appears all mice were given food soaked in water, which was spread on different places on the bedding of the cages. For calculation purposes, the score of animals that were sacrificed or died (6) was carried forward.

Interpretation of Results

Calculation of the Incidence of Disease (Disease Ratio)
The number of sick animals in each group were summed.
The incidence of disease was calculated as

$$INCIDENCE of DISEASE = ( \frac{No . of sick mice in treated group}{No . of sick mice in control group} )$$

The percent inhibition according to incidence was calculated as

$$INHIBITION ( \% ) of INCIDENCE = ( 1 - \frac{Number of sick mice in treated group}{Number of sick mice in control group} ) \times 100$$

Calculation of the Mortality/Moribundity Rate (Mortality Ratio)
The number of dead or moribund animals in each group were summed.
The mortality of disease was calculated as

$$MORTALITY of DISEASE = ( \frac{No . of dead or moribound mice in treated group}{No . of dead or moribound mice in control group} )$$

The percent inhibition according to mortality was calculated as

$$INHIBITION ( \% ) of MORTALITY = ( 1 - \frac{Number of dead or moribound mice in treated group}{Number of dead or moribound mice in control group} ) \times 100$$

Calculation of Duration of Disease
The mean duration of disease expressed in days was calculated as

$$Mean Duration = ( \frac{Σ Duration of disease of each mouse}{No . of mice in the group} )$$

Calculation of Mean Delay in Onset of Disease
The mean onset of disease expressed in days was calculated as

$$Mean Onset = ( \frac{Σ Onset of disease of each mouse}{No . of mice in the group} )$$

The mean delay in onset of disease expressed in days was calculated by subtracting the mean onset of disease in control group from test group.

Calculation of the Mean Maximal Score and Percent Inhibition
The mean maximal score (MMS) of each group was calculated as

$$MMS = ( \frac{Σ Maximal Score of each mouse}{No . of mice in the group} )$$

The percent inhibition according to MMS was calculated as

$$INHIBITION ( \% ) of MMS = ( 1 - \frac{MMS of treated group}{MMS of control group} ) \times 100$$

Calculation of the Group Mean Score and Percent Inhibition
The daily scores of each mouse in the test group were summed and the individual mean daily score (IMS) was calculated as

$$IMS = ( \frac{Σ Daily score of mouse}{Observation period ( days )} )$$

The mean group score (GMS) was calculated as

$$GMS = ( \frac{Σ IMS of each mouse}{No . of mice in the group} )$$

The percent inhibition was calculated as

$$INHIBITION ( \% ) of GMS = ( 1 - \frac{GMS of treated group}{GMS of control group} ) \times 100$$

Results/Discussion

A summary of the incidence, mortality, mean maximal scores (MMS), group mean score (GMS), duration of the disease, onset of the disease and the activity of each group compared to the vehicle treated control group is shown in Table 3 below.

TABLE 3
Mortality, incidence, MMS, GMS, Duration and Onset and EAE inhibition compared to Vehicle

| | | | | MMS | | GMS | | Mean Onset | Mean Duration |
|---|---|---|---|---|---|---|---|---|---|
| Treatment | Mortality | Incidence | % inhibition 1 | value | % inhibition 2 | value | % inhibition 3 | (days) | (days) |
| Negative | 0/13 | 13/13 | — | 3.2 ± 1.0 | — | 2.0 ± 1.0 | — | | |
| Control | | | | | | | | | |
| Vehicle 10 | | | | | | | | | |
| Laquinimod | 0/13 | 8/13 | 38.5% | 1.8 ± 1.7 | 43.8% | 0.8 ± 0.8 | 60.0% | 21.3 ± 8.2 | 9.2 ± 8.3 |
| 10 mg/kg | | | | | p = 0.04 | | p = 0.003 | p = <0.001 | p = |
| Laquiniomd | 0/15 | 5/15 | 61.5% | 0.8 ± 1.3 | 75.0% | 0.3 ± 0.5 | 85.0% | 25.7 ± 7.8 | 5.0 ± 7.4 |
| 25 mg/kg | | | | | p < 0.001 | | | p = <0.001 | p = < |
| IFN-β | 0/13 | 11/13 | 15.4% | 2.6 ± 1.4 | 18.8% | 1.7 ± 1.0 | 15.0% | 14.9 ± 7.3 | 15.4 ± 7.8 |
| 50,000 | | | | | P = 0.2 | | p = 0.3 | p = 0.003 | p = 0.3 |
| IU/mouse | | | | | | | | | |
| IFN-β | 0/13 | 12/13 | 7.7% | 1.7 ± 1.0 | 46.9% | 0.9 ± 0.7 | 55.0% | 14.7 ± 5.1 | 13.9 ± 6.1 |
| 500,000 | | | | | p = 0.001 | | p = 0.007 | p < 0.001 | p = 0.01 |
| IU/mouse | | | | | | | (p < 0.001) | | |
| Laquinimod + | 0/13 | 4/13 | 69.2% | 1.0 ± 1.7 | 68.8% | 0.5 ± 0.9 | 75.0% | 26.4 ± 7.3 | 17.6 ± 2.7 |
| IFN-β | | | | | p = 0.003 | | p = 0.001 | p = <0.001 | p = 0.3 |
| 10 mg/kg + | | | | | | | | | |
| 50,000 | | | | | | | | | |
| IU/mouse | | | | | | | | | |
| Laquinimod + | 0/15 | 5/15 | 61.5% | 0.8 ± 1.2 | 75.0% | 0.2 ± 0.3 | 90.0% | 27.6 ± 5.4 | 3.4 ± 5.4 |
| IFN-β | | | | | P < 0.001 | | P < 0.001 | P < 0.001 | p < 0.001 |
| 10 mg/kg + | | | | | | | (p < 0.001) | | |
| 500,000 | | | | | | | | | |
| IU/mouse | | | | | | | | | |
| IFN-β (Day 1 | 0/13 | 13/13 | 0% | 3.1 ± 0.3 | 3.1% | 1.7 ± 0.4 | 15.0% | 12.9 ± 1.3 | 17.5 ± 1.7 |
| to 7) | | | | | p = 0.4 | | p = 0.4 | p < 0.001 | p = 0.09 |
| 500,000 | | | | | | | | | |
| IU/mouse | | | | | | | | | |
| IFN-β (Day 8 | 0/13 | 10/13 | 23.1% | 2.3 ± 1.7 | 28.1% | 1.3 ± 1.1 | 35.0% | 16.5 ± 8.4 | 12.2 ± 8.7 |
| to 30) | | | | | p = 0.1 | | p = 0.05 | p = 0.008 | p = 0.04 |
| 500,000 | | | | | | | (p < 0.001) | | |
| IU/mouse | | | | | | | | | |

The activity of the IFN-β administered groups in combination with laquinimod (10 mg/kg) compared to the group treated with laquinimod (10 mg/kg) is shown in Table 4 below.

TABLE 4
Laquinimod alone and in combination with IFN-β compared to laquinimod (10 mg/kg).

| | | | | | | | | Mean Onset | Mean Duration |
|---|---|---|---|---|---|---|---|---|---|
| Treatment | Mortality | Incidence | % inhibition 1 | MMS | % inhibition 2 | GMS | % inhibition 3 | (days) | (days) |
| Laquinmod | 0/13 | 8/13 | — | 1.8 ± 1.7 | — | 0.8 ± 0.8 | — | 21.4 ± 8.2 | 9.2 ± 8.3 |
| 10 mg/kg | | | | | | | | | |
| Laquinmod | 0/15 | 5/15 | 37.5% | 0.8 ± 1.3 | 55.5% | 0.3 ± 0.5 | 62.5% | 25.7 ± 7.8 | 5.0 ± 7.4 |
| 25 mg/kg | | | | | p = 0.17 | | p = 0.10 | p = 0.29 | p = 0.24 |
| Laquinmod + | 0/13 | 4/13 | 50.0% | 1.0 ± 1.7 | 44.4% | 0.5 ± 0.9 | 37.5% | 26.5 ± 7.3 | 17.6 ± 2.7 |
| IFN-β | | | | | p = 0.26 | | p = 0.31 | p = 0.15 | p = 0.003 |
| 10 mg/kg + | | | | | | | | | |
| 50,000IU/mouse | | | | | | | | | |
| Laquinmod + | 0/15 | 5/15 | 37.5% | 0.8 ± 1.2 | 55.5% | 0.2 ± 0.3 | 75.0% | 27.6 ± 5.4 | 3.4 ± 5.4 |
| IFN-β | | | | | P = 0.13 | | P = 0.10 | P = 0.081 | p = 0.091 |
| 10 mg/kg + | | | | | | | (p < 0.001) | | |
| 500,000 | | | | | | | | | |
| IU/mouse | | | | | | | | | |

The activity as compared to vehicle and laquinimod are shown in Tables 5 and 6 below:

TABLE 5
Activity compared to vehicle

| | Group Mean Score (GMS) | Activity |
|---|---|---|
| Vehicle (saline) | 2.0 | |
| Laquinimod (10 mg/kg) | 0.8 | 60% |
| Laquinimod (25 mg/kg) | 0.3 | 85% |
| IFN beta (50,000 IU/mouse) | 1.7 | 15% |
| IFN beta (500,000 IU/mouse) | 0.9 | 55% |
| IFN beta (50,000 IU/mouse) | 0.5 | 75% |
| Laquinimod (10 mg/kg) | | |
| IFN beta (500,000 IU/mouse) + | 0.2 | 90% |
| Laquinimod (10 mg/kg) | | |

TABLE 6
Activity compared to Laquinimod

| | Group Mean Score (GMS) | Activity |
|---|---|---|
| Vehicle (saline) | 2.0 | |
| Laquinimod (10 mg/kg) | 0.8 | 60% |
| Laquinimod (25 mg/kg) | 0.3 | 85% |
| IFN beta (50,000 IU/mouse) | 1.7 | 15% |
| IFN beta (500,000 IU/mouse) | 0.9 | 55% |

TABLE 7
Activity compared to interferon-β (500,000 IU/mouse)

| | Group Mean Score (GMS) | Activity |
|---|---|---|
| IFN beta (500,000 IU/mouse) | 0.9 | — |
| Laquinimod (25 mg/kg) | 0.3 | 66.7% |
| IFN beta (50,000 IU/mouse) + | 0.5 | 44% |
| Laquinimod (10 mg/kg) | | |
| IFN beta (500,000 IU/mouse) + | 0.2 | 78% |
| Laquinimod (10 mg/kg) | | |
| IFN beta (500,000 IU/mouse) | 0.9 | — |

Under the conditions of the test IFN-β at dose levels of 50,000 IU/mouse and 500,000 IU/mouse exhibited additive activity in the suppression of EAE when tested in combination with laquinimod at dose level of 10 mg/kg.

The groups treated with IFN-β at dose levels of 50,000 IU/mouse and 500,000 IU/mouse in combination with laquinimod (10 mg/kg) exhibited 75% and 90% activity respectively according to GMS compared to 15%, 55% and 60% activity in groups treated with IFN-β at dose levels of 50,000 IU/mouse and 500,000 IU/mouse and laquinimod at dose level of 10 mg/kg respectively when compared to the vehicle administered control group.

The groups treated with IFN-β at dose levels of 50,000 IU/mouse and 500,000 IU/mouse in combination with laquinimod (10 mg/kg) exhibited 37.5% and 75% activity respectively according to GMS when compared to group treated with laquinimod at dose level of 10 mg/kg.

It is important to note the mouse dosing presented here cannot be used to determine human dosing by simply adjusting for body weight, because a gram of mouse tissue is not equivalent to a gram of human tissue. For this reason, the National Institutes of Health (NIH) provides a table of Equivalent Surface Area Dosage Conversion Factors below (Table 8) which provides conversion factors that account for surface area to weight ratios between species.

TABLE 8
Equivalent Surface Area Dosage Conversion Factors

| | To | | | | |
|---|---|---|---|---|---|
| FROM | Mouse 20 g | Rat 150 g | Monkey 3 kg | Dog 8 kg | Man 60 kg |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | 1⅔ | 1 | ½ |
| Man | 12 | 7 | 3 | 2 | 1 |

### Example 3

### Clinical Trial (Phase II)—Assessment Of Add-on Effect Of Laquinimod In Relapsing Multiple Sclerosis (RMS) Subjects Treated With Glatiramer Acetate (GA) Or Interferon-beta (IFN-β)

A multinational, multicenter, randomized, double-blind, parallel-group, placebo-controlled study, followed by a double-blind active extension phase is conducted to assess the safety, tolerability and efficacy of two daily doses of oral laquinimod (0.6 mg or 1.2 mg) in adjunct to glatiramer acetate (GA) or interferon-beta (IFN-β)-1a/1b preparations in subjects with relapsing multiple sclerosis (RMS).

Study Duration

The total study duration for each eligible subject will be up to 19 months:
Screening phase: up to about 1 month.
Double-Blind Placebo Controlled (DBPC) treatment phase: about 9 months of once-daily oral administration of laquinimod 0.6 mg/day, 1.2 mg/day or placebo in addition to current therapy (i.e., subcutaneous GA 20mg or any of the following IFN-β preparations: Avonex®, Betaseron®/Betaferon®, Rebif® or Extavia®).
Double-Blind Active Extension (DBAE) phase: all subjects who complete all 9 months of the DBPC treatment phase are offered the opportunity to continue to a DBAE phase. During this phase, all subjects continue the same background injectable treatment which they used in the DBPC phase.
Subjects who were originally assigned to either of the active oral treatment arms (laquinimod 0.6 mg or 1.2 mg) continue with their original oral treatment assignment. Subjects originally assigned to placebo are equally randomized to either laquinimod 0.6 mg or 1.2 mg. The duration of this phase is 9 months.

Study Population

Relapsing Multiple Sclerosis (RMS).

### Study Design

Eligible subjects are equally (1:1:1) randomized into one of the following treatment arms:

1. GA 20 mg or any IFN-β preparation+oral daily administration of laquinimod capsules 0.6 mg.

2. GA 20 mg or any IFN-β preparation+oral daily administration of laquinimod capsules 1.2 mg.

3. GA 20 mg or any IFN-β preparation +oral daily placebo .

The 0.6 mg laquinimod capsule can be manufactured according to the method disclosed in PCT International Application Publication No. WO/2007/146248, published Dec. 21, 2007 (see, page 10, line 5 to page 11, line 3).

Randomization is stratified in a way that in each arm the number of subjects treated by GA will be equal to the number of subjects treated by IFN-β preparations (Avonex®, Betaseron /Betaferon®, Rebif®or Extavia®).

During the DBAE phase, subjects continue the same background injectable treatment which they used in the DBPC phase. Subjects who were originally assigned to either of the active oral arms [either laquinimod 0.6 mg (arm 1) or 1.2 mg (arm 2)] continue with their original oral treatment assignment. Subjects originally assigned to placebo (arm 3) are equally randomized to either laquinimod 0.6 mg or 1.2 mg.

During the DBPC phase, subjects are evaluated at study sites for 11 scheduled visits at Months: −1 (screening), 0 (baseline) and every month thereafter until Month 9 (termination/early termination).

During the DBAE phase subjects are evaluated at study sites for 6 scheduled visits at months 9 [Baseline EXT; the termination visit of the DBPC phase], 10/1AE, 11/2AE, 12/3AE, 15/4AE and 18/5AE (termination/early termination visit of the DBAE phase).

The following assessments are performed at the specified time points:

1. During both DBPC and DBAE phases, vital signs are measured at each study visit.

2. During the DBPC phase, a physical examination is performed at Month—1 (Screening) and Months 0 (Baseline), 1, 3, 6 and 9 (Termination/Early Termination visit of the DBPC phase). During the DBAE phase, a physical examination is performed at Month 9 (Baseline EXT; termination visit of the DBPC phase), 10/1AE, 12/3AE and 18/5AE (Termination/Early Termination visit of the DBAE phase).

3. The following safety clinical laboratory tests are performed:
a. Complete blood count (CBC) with differential—at all scheduled visits in both DBPC and DBAE phases.
b. Serum chemistry (including electrolytes, liver enzymes, creatinine, direct and total bilirubin and pancreatic amylase), and urinalysis is performed at all scheduled visits in both DBPC and DBAE phases. Lipase is tested in case of abnormal pancreatic amylase results. Glomerular filtration rate (GFR) is calculated at Month—1 (Screening) and prior to each MRI scan.
c. Lipid profile (total cholesterol, HDL, LDL and triglycerides) is performed at month—1 (Screening) or Month 0 (Baseline) of the DBPC phase, under fasting conditions.
d. During the DBPC phase, Thyroid function tests (TSH, T3 and free T4) are performed at Months 0 (Baseline), 6 and 9 (Termination/Early termination visit of the DBPC phase). During the DBAE phase thyroid function tests (TSH, T3 and free T4) are performed at Months 9 (Baseline EXT; termination visit of the DBPC phase), 15/4AE and 18/5AE (termination/ early termination visit of the DBAE phase).
e. Urinalysis is performed at the Screening visit.
f. Serum β-hCG (human choriogonadotropin beta) is performed in women of child-bearing potential at each scheduled study visit in both DBPC and DBAE phases.

4. Urine dipstick β-hCG in women of child-bearing potential during both the DBPC and the DBAE phases, at all post-Screening study visits and the early termination visit. In addition, during the DBAE Phase, urine β-hCG test is performed at home twice between scheduled visits:
a. At months 13AE and 14AE (30±4 days and 60±4 after Month 12AE visit, respectively).
b. At months 16AE and 17AE (30±4 days and 60±4 after Month 15AE visit, respectively).

The subject is contacted by the site staff via telephone within 72 hours after the test is scheduled to be performed and asked specific questions regarding the test. In case of suspected pregnancy (positive urine β-hCG test result), the caller instructs the subject to stop taking the study drug and to arrive to the site as soon as possible (but within 10 days) with all study drugs.

5. During the DBPC phase, electrocardiograms (ECG) is performed at months—1 (screening), 0 (Baseline; three recordings 10 min apart, before first dose), 1, 2, 3, 6 and 9 (termination/early termination visit of the DBPC phase). During the DBAE phase, ECGs are performed at Months 9 (Baseline EXT; termination visit of the DBPC phase), 10/1AE, 11/2AE, 12/3AE, 15/4AE and 18/5AE (Termination/ Early Termination visit of the DBAE phase).

6. Chest X-ray is performed at month—1 (screening), if not performed within 6 months prior to the screening visit.

7. Adverse Events (AEs) are monitored throughout the study.

8. Concomitant Medications are monitored throughout the study (both phases).

9. During the DBPC phase, neurological evaluations, including Expanded Disability Status Scale (EDSS), Ambulation Index (AI) and Functional system score (FS) are performed at Months:—1 (screening), 0 (baseline), 3, 6, and 9 (Termination/Early Termination of the DBPC phase). During the DBAE phase, neurological evaluations, including EDSS, AI and FS scores are performed at Months 9 (Baseline; termination visit of the DBPC phase), 12/3AE, 15/4AE and 18/5AE (Termination/Early Termination of the DBAE phase).

10. During the DBPC phase, Symbol Digit Modalities Test (SDMT) are performed at Months 0 (Baseline), 6 and 9 (Termination/Early Termination visit of the DBPC phase). During the DBAE phase, SDMT is performed at Months 9 (Baseline EXT; Termination visit of the DBPC phase), 15/4AE and 18/5AE (Termination/Early Termination visit of the DBAE phase).

11. During the DBPC phase, each subject undergoes 3 MRI scans at Months: 0 (baseline), 3 and 9 (Termination/Early Termination visit of the DBPC phase). During the DBAE phase, each subject undergoes 2 MRI scans at Months 9 (Baseline EXT; Termination visit scan of the DBPC phase) and 18/5AE (Termination/Early Termination visit of the DBAE phase).

12. During the DBPC phase, Pharmacokinetic (PK) study: Blood samples for analysis of laquinimod plasma concentrations are collected from all subjects at Months 1, 3 and 6.

13. During the DBPC phase, whole blood samples are collected for lymphocyte immunophenotyping at Months 0 (Baseline), 3 and 9 (Termination/Early Termination).

14. Health Economics and Quality of Life: During the DBPC phase, the Work Productivity and Activities Impairment Questionnaire-General Health (WPAI-GH) (US sites only) and the European Quality of Life (EuroQoL) Questionnaire (EQ5D) are filled out at months 0 (Baseline), and 9 (Termination/Early Termination). During the DBAE phase, the WPAI-GH (US sites only) and the EQ5D questionnaire are filled out at Months 9 (Baseline EXT; Termination visit of the DBPC phase) and 18/5AE (termination/early termination visit of the DBAE phase).

15. Relapses are confirmed/monitored throughout the study (both phases).

### Relapse Treatment

The allowed treatment for a relapse is intravenous Methylprednisolone 1 gr/day for up to 5 consecutive days.

### Monitoring

The subjects are closely monitored through the study course by an external independent Data Monitoring Committee (DMC).

### MRI Activity Alert Criteria

In case 5 or more GdE-T1 lesions are demonstrated on an MRI scan, the MRI reading center issues a notification letter to the Sponsor, investigator and the DMC. MRI parameters of activity are not considered stopping rules and the decision regarding individual subject's participation in the trial is at the discretion of the treating physician.

### Ancillary Studies

Pharmacogenetic (PGx) assessment: Blood samples for PGx parameters are collected from all subjects that signed the informed consent form (separate from that of the core study), pending Ethics Committees approval, during the DBPC phase, preferably at Month 0 (Baseline) or any other visit following Month 0.

Number of Subjects

Approximately 600 subjects.

Inclusion/Exclusion Criteria

### Inclusion Criteria 1. Subjects must have a documented MS diagnosis as defined by the Revised McDonald criteria [Ann Neurol 2011: 69:292-302], with a relapsing disease course.

2. Subjects must be relapse free, in a stable neurological condition and free of corticosteroid treatment [intravenous (IV), intramuscular (IM) and/or oral] 60 days prior to randomization.

3. Subjects must be treated with GA (Copaxone®) or an IFN-β preparation (Avonex®, Betaseron/Betaferon , Rebif® or Extavia), at a stable dose for at least 6 months prior to randomization (switching between IFN-β preparations during the 6 months prior to randomization is allowed; switching between any IFN-β preparation and GA, or vice versa, is exclusionary), and there is no plan to change the subject's injectable treatment (either Copaxone® or IFN-β preparation) during the course of the study.

4. Subjects must have an EDSS score of 1.5-4.5 (inclusive) at randomization.

5. Subjects must be between 18 and 55 years of age, inclusive.

6. Women of child-bearing potential must practice an acceptable method of birth control. Acceptable methods of birth control in this study include: surgical sterilization, intrauterine devices, oral contraceptive, contraceptive patch, long-acting injectable contraceptive, partner's vasectomy or double-barrier method (condom or diaphragm with spermicide).

7. Subjects must be able to sign and date a written informed consent prior to entering the study.

8. Subjects must be willing and able to comply with the protocol requirements for the duration of the study.

### Exclusion Criteria

1. Have a non-relapsing, progressive form of MS (e.g., PPMS) (as defined by Lublin and Reingold, 1996).

2. An onset of a relapse, unstable neurological condition or any treatment with corticosteroids [intravenous (iv), intramuscular (im) and/or per os (po)] or Adrenocorticotropic hormone 60 days prior to randomization (last day of steroid treatment should be equal or greater than 60 days prior to randomization).

3. Use of experimental or investigational drugs, and/or participation in drug clinical studies within the 6 months prior to randomization.

4. Use of immunosuppressive agents within 6 months prior to randomization.

5. Use of natalizumab (Tysabri®), fingolimod (Gilenya®) or anti-B cell therapy within the 2 years prior to randomization.

6. Previous use of any of the following: cytotoxic agents, Mitoxantrone (Novantrone®), cladribine, laquinimod, total body irradiation, total lymphoid irradiation, stem cell treatment, autologous bone marrow transplantation or allogenic bone marrow transplantation.

7. Previous treatment with intravenous immunoglobulin (IVIG) or plasmapheresis within 2 months prior to randomization.

8. Use of moderate/strong inhibitors of CYP3A4 within 2 weeks prior to the randomization

9. Use of inducers of CYP3A4 within 2 weeks prior to randomization.

10. Pregnancy or breastfeeding.

11. A ≧2×ULN serum elevation of either alanine transaminase (ALT) or aspartate transaminase (AST) at screening.

12. Serum direct bilirubin which is ≧2×ULN at screening

13. Subjects with a potentially clinically significant or unstable medical or surgical condition that would preclude safe and complete study participation, as determined by medical history, physical examinations, ECG, laboratory tests or chest X-ray. Such conditions may include:
a. A cardiovascular or pulmonary disorder that cannot be well-controlled by standard treatment permitted by the study protocol.
b. Renal diseases.
c. Any form of acute or chronic liver disease.
d. Known human immunodeficiency virus (HIV) positive status.
e. A history of drug and/or alcohol abuse.
f. An unstable psychiatric disorder.
g. Any malignancies, excluding basal cell carcinoma (BCC), in the last 5 years.

14. A glomerular filtration rate (GFR) less than 60 ml/min at screening visit.

15. A known history of sensitivity to gadolinium (Gd).

16. Inability to successfully undergo MRI scanning

17. Previous endovascular treatment for Chronic Cerebrospinal Venous Insufficiency (CCSVI).

18. Known drug hypersensitivity that would preclude administration of laquinimod, such as hypersensitivity to: mannitol, meglumine or sodium stearyl fumarate.

Route and Dosage Form

1. GA 20 mg or an preparation of interferon-beta (IFN-β)+oral daily administration of laquinimod capsules 0.6 mg (one laquinimod capsule 0.6 mg and one placebo capsule for laquinimod) (applicable to both DBPC and DBAE phases).

2. GA 20 mg/1 mL or an IFN-β preparation+oral daily administration of laquinimod 1.2 mg (2 capsules of laquinimod 0.6 mg) (applicable to both DBPC and DBAE phases)

3. GA 20 mg or an preparation of IFN-β oral daily administration of placebo (2 placebo capsules for laquinimod) (applicable only to DBPC phase).

Outcome Measures

The primary objectives of the study are to assess the safety, tolerability and efficacy of two daily doses of oral laquinimod (0.6 mg or 1.2 mg) in adjunct to GA or IFN-β preparation (Avonex®, Betaseron®/Betaferon®, Rebif® or Extavia®) in subjects with RMS.

Primary Efficacy Endpoint for DBPC Phase:
The percent brain volume change (PBVC) between month 0 (Baseline) to Month 9 (Termination/Early Termination after Month 6 of the DBPC phase).

### Key Exploratory Efficacy Endpoints for DBPC Phase

Change in whole brain Magnetic Transfer Ratio (MTR) histogram between month 0 (Baseline) and Month 9 (Termination/Early Termination visit after Month 6 of the DBPC phase).
Time to Confirmed Disease Progression (CDP). CDP is defined as a sustained increase in EDSS of ≧1 point from Baseline for at least 3 months. Progression cannot be confirmed during a relapse.

### Exploratory Endpoints for DBPC Phase

The percent change in cortical thickness between month 0 (baseline) and month 9 (termination/early termination visit after month 6).
The cumulative number new T1 hypointense lesions at months 3 and 9 (termination/early termination visit after month 6).
The number of active (new T2 or GdE-T1) lesions at month 3 that evolved into black holes at month 9 (termination/early termination visit after month 6).
The cumulative number of GdE-T1 lesions at months 3 and 9 (termination/early termination visit after month 6).
Change in T2 lesion volume from 0 (baseline) to month 9 (termination/early termination visit after month 6).
Change in GdE-T1 lesions volume from month 0 (baseline) to month 9 (termination/early termination visit after month 6).
Change from baseline to month 9 (termination/early termination visit after month 6) in SDMT score.
The general health status, as assessed by the EuroQoL (EQ5D) questionnaire.
Assessment of the effect of general health and symptom severity on work, using the work productivity and activities impairment General Health (WPAI-GH) questionnaire.
Annualized Relapse Rate (ARR).
The time to the first confirmed relapse.
Pharmacokinetics of laquinimod.

### Exploratory Endpoints for DBAE Phase

A similar set of endpoints are analyzed for the DBAE phase.

### Safety and Tolerability Endpoints for DPBC phase

The cumulative number of GdE-T1 lesions at months 3 and 9.
The cumulative number of Combined Unique Active (CUA) lesions at months 3 and 9.
Number of subjects with adverse events.
Number of subjects with potentially clinically significant abnormalities based on laboratory tests and vital signs and ECGs during the study.
Proportion of subjects (%) who prematurely discontinue from the study, reason of discontinuation and the time to withdrawal.
Proportion of subjects (%) who prematurely discontinue from the study due to adverse events (AEs) and the time to withdrawal.

Results/Discussion

This study assesses safety, tolerability and efficacy of laquinimod in adjunct to glatiramer acetate

(GA) or interferon-beta (IFN-β ) in relapsing multiple sclerosis (RMS) subjects. Since the mechanisms of action of laquinimod and IFN-β have not been fully elucidated, the effect of the combined therapy cannot be predicted and must be evaluated experimentally.

Daily administration of laquinimod (p.o., 0.6 mg/day and 1.2 mg/day) as an add-on therapy for a patient already receiving interferon-beta (IFN-β ) provides increased efficacy (provides an additive effect or more than an additive effect) in relapsing multiple sclerosis (RMS) subjects without unduly increasing adverse side effects or affecting the safety of the treatment. Daily administration of laquinimod (p.o., 0.6 mg/day and 1.2 mg/day) as an add-on therapy to IFN-β is also safe for use in treating relapsing multiple sclerosis (RMS) patients.

Administration of laquinimod (p.o., 0.6 mg/day and 1.2 mg/day) as an add-on therapy to IFN-β provides a clinically meaningful advantage and is more effective (provides an additive effect or more than an additive effect) in treating relapsing multiple sclerosis (RMS) patients than when IFN-β is administered alone (at the same dose) in the following manner:

1. The add-on therapy is more effective (provides an additive effect or more than an additive effect) in reducing the decrease in brain volume (determined by the percent brain volume change (PBVC)), in relapsing multiple sclerosis (RMS) patients.

2. The add-on therapy is more effective (provides an additive effect or more than an additive effect) in increasing the time to confirmed disease progression (CDP), in relapsing multiple sclerosis (RMS) patients, where CDP is defined as a sustained increase in EDSS of 1 point from Baseline for at least 3 months. Progression cannot be confirmed during a relapse.

3. The add-on therapy is more effective (provides an additive effect or more than an additive effect) in reducing abnormalities observed in whole Brain MTR histogram, in relapsing multiple sclerosis (RMS) patients during.

4. The add-on therapy is more effective (provides an additive effect or more than an additive effect) in reducing the number of confirmed relapses and therefore the relapse rate, in relapsing multiple sclerosis (RMS) patients.

5. The add-on therapy is also more effective (provides an additive effect or more than an additive effect) in reducing the accumulation of physical disability in relapsing multiple sclerosis (RMS) patients, as measured by the time to confirmed progression of EDSS.

6. The add-on therapy is more effective (provides an additive effect or more than an additive effect) in reducing MRI-monitored disease activity in relapsing multiple sclerosis (RMS) patients, as measured by the cumulative number of T1 Gd-enhancing lesions on T1-weighted images, the cumulative number new T1 hypointense lesions, the cumulative number of new T2 lesions, the cumulative number of new T1 hypointense lesions on T1-weight images (black holes), the number of active (new T2 or GdE-T1) lesions, presence or absence of GdE lesions, change in total volume of T1 Gd-enhancing lesions, change in total volume of T2 lesions, and/or cortical thickness.

7. The add-on therapy is more effective (provides an additive effect or more than an additive effect) in reducing brain atrophy in relapsing multiple sclerosis (RMS) patients.

8. The add-on therapy is more effective (provides an additive effect or more than an additive effect) in reducing the frequency of relapses, the frequency of clinical exacerbation, and the risk for confirmed progression in relapsing multiple sclerosis (RMS) patients.

9. The add-on therapy is more effective (provides an additive effect or more than an additive effect) in increasing the time to confirmed relapse in relapsing multiple sclerosis (RMS) patients.

10. The add-on therapy is more effective (provides an additive effect or more than an additive effect) in improving the general health status (as assessed by the EuroQoL (EQ5D) questionnaire), symptom severity on work (as assessed by the work productivity and activities impairment General Health (WPAI-GH) questionnaire) and quality of life, in relapsing multiple sclerosis (RMS) patients.

11. The add-on therapy is more effective (provides an additive effect or more than an additive effect) in decreasing cerebral dysfunction/cognitive impairment (as assessed by Symbol Digit Modalities Test (SDMT)), in relapsing multiple sclerosis (RMS) patients during the double blind study period.

Administration of laquinimod (p.o., 0.6 mg/day and 1.2 mg/day) as an add-on therapy to IFN-β provides a clinically meaningful advantage and is more effective (provides an additive effect or more than an additive effect) in delaying the conversion to clinically definite MS in patients presenting a CIS suggestive of MS than when IFN-β is administered alone (at the same dose).

Administration of laquinimod (p.o., 0.6 mg/day and 1.2 mg/day) as an add-on therapy to IFN-β provides a clinically meaningful advantage and is more effective (provides an additive effect or more than an additive effect) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when IFN-β is administered alone (at the same dose).

Based on the foregoing, analogous results are expected for therapy using laquinimod (p.o., 0.6 mg/day and 1.2 mg/day) in combination with IFN-. Specifically, daily administration of laquinimod (p.o., 0.6 mg/day and 1.2 mg/day) in combination with IFN-β provides increased efficacy (provides an additive effect or more than an additive effect) over the administration of each agent alone in relapsing multiple sclerosis (RMS) subjects without unduly increasing adverse side effects or affecting the safety of the treatment. Daily administration of laquinimod (p.o., 0.6 mg/day) in combination with interferon-beta (IFN-β) is also safe for use in treating relapsing multiple sclerosis (RMS) patients.

Administration of laquinimod (p.o., 0.6 mg/day and 1.2 mg/day) in combination with IFN-β provides a clinically meaningful advantage and is more effective (provides an additive effect or more than an additive effect) in treating relapsing multiple sclerosis (RMS) patients than when IFN-β is administered alone (at the same dose) in the following manner:

12. The combination therapy is more effective (provides an additive effect or more than an additive effect) in reducing the decrease in brain volume (determined by the percent brain volume change (PBVC)), in relapsing multiple sclerosis (RMS) patients.

13. The combination therapy is more effective (provides an additive effect or more than an additive effect) in increasing the time to confirmed disease progression (CDP), in relapsing multiple sclerosis (RMS) patients, where CDP is defined as a sustained increase in EDSS of ≧1 point from Baseline for at least 3 months. Progression cannot be confirmed during a relapse.

14. The combination therapy is more effective (provides an additive effect or more than an additive effect) in reducing abnormalities observed in whole Brain MTR histogram, in relapsing multiple sclerosis (RMS) patients during.

15. The combination therapy is more effective (provides an additive effect or more than an additive effect) in reducing the number of confirmed relapses and therefore the relapse rate, in relapsing multiple sclerosis (RMS) patients.

16. The combination therapy is also more effective (provides an additive effect or more than an additive effect) in reducing the accumulation of physical disability in relapsing multiple sclerosis (RMS) patients, as measured by the time to confirmed progression of EDSS.

17. The combination therapy is more effective (provides an additive effect or more than an additive effect) in reducing MRI-monitored disease activity in relapsing multiple sclerosis (RMS) patients, as measured by the cumulative number of T1 Gd-enhancing lesions on T1-weighted images, the cumulative number new T1 hypointense lesions, the cumulative number of new T2 lesions, the cumulative number of new T1 hypointense lesions on T1-weight images (black holes), the number of active (new T2 or GdE-T1) lesions, presence or absence of GdE lesions, change in total volume of T1 Gd-enhancing lesions, change in total volume of T2 lesions, and/or cortical thickness.

18. The combination therapy is more effective (provides an additive effect or more than an additive effect) in reducing brain atrophy in relapsing multiple sclerosis (RMS) patients.

19. The combination therapy is more effective (provides an additive effect or more than an additive effect) in reducing the frequency of relapses, the frequency of clinical exacerbation, and the risk for confirmed progression in relapsing multiple sclerosis (RMS) patients.

20. The combination therapy is more effective (provides an additive effect or more than an additive effect) in increasing the time to confirmed relapse in relapsing multiple sclerosis (RMS) patients.

21. The combination therapy is more effective (provides an additive effect or more than an additive effect) in improving the general health status (as assessed by the EuroQoL (EQ5D) questionnaire), symptom severity on work (as assessed by the work productivity and activities impairment General Health (WPAI-GH) questionnaire) and quality of life, in relapsing multiple sclerosis (RMS) patients.

22. The combination therapy is more effective (provides an additive effect or more than an additive effect) in decreasing cerebral dysfunction/cognitive impairment (as assessed by Symbol Digit Modalities Test (SDMT)), in relapsing multiple sclerosis (RMS) patients during the double blind study period.

Administration of laquinimod (p.o., 0.6 mg/day and 1.2 mg/day) in combination with IFN-β provides a clinically meaningful advantage and is more effective (provides an additive effect or more than an additive effect) in delaying the conversion to clinically definite MS in patients presenting a CIS suggestive of MS than when IFN-β is administered alone (at the same dose).

Administration of laquinimod (p.o., 0.6 mg/day and 1.2 mg/day) in combination with IFN-β provides a clinically meaningful advantage and is more effective (provides an additive effect or more than an additive effect) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when IFN-β is administered alone (at the same dose).

### REFERENCES

1. “COPAXONE®” in Physician's Desk Reference, Thompson Reuters—Physician's Desk Reference Inc., Montvale, N.J., 2008, 3110-3113.

2. Barkhof, F. (1999) “MRI in Multiple Sclerosis: Correlation with Expanded Disability Status Scale (EDSS)”, Multiple Sclerosis. 5(4):283-286 (Abstract).

3. Bjartmar C, Fox R I. (2002) “Pathological mechanisms and disease progression of multiple sclerosis: therapeutic implication”, Drugs of Today. 38:7-29.

4. Brex et al., (2002) “A longitudinal study of abnormalities on MRI and disability from multiple sclerosis”, N Engl J Med. Jan. 17, 2002 346(3):158-64.

5. Brod et al. (2000) Annals of Neurology, 47:127-131.

6. Brück (2011) “Insight into the mechanism of laquinimod action.” J Neurol Sci. 2011 Jul. 15;306(1-2):173-9.

7. Brunmark et al., (2002) “The new orally active immunoregulator laquinimod (ABR-215062) effectively inhibits development and relapses of experimental autoimmune encephalomyelitis”, J Neuroimmunology. 130:163-172.

8. Chabot and Yong, Interferon-lb increases IL-10 in a model of T cell—microglia interaction: Relevance to MS, Neurol. 2000, 55: 1497-1505.

9. Chabot et al., Cytokine production in T lymphocyte-microglia interaction is attenuated by glatiramer acetate: A mechanism for therapeutic efficacy in multiple sclerosis, Mult. Scler., in press.

10. Comi et al. (2007) LAQ/5062 Study Group. “The Effect of Two Doses of Laquinimod on MRI-Monitored Disease Activity in Patients with Relapsing-Remitting Multiple Sclerosis: A Multi-Center, Randomized, Double-Blind, Placebo-Controlled Study”, Presented at: 59th Annual Meeting of the American Academy of Neurology; Apr. 28-May 5, 2007; Boston, Mass.

11. Compston, Genetic susceptibility to multiple sclerosis, in McAlpine's Mutiple Sclerosis, Matthews, B. ed., London: Churchill Livingstone, 1991, 301-319.

12. Conway and Cohen (2010) “Combination therapy in multiple sclerosis”, LancetNeurol, 9:299-308.

13. Costello et al. (2007) “Combination therapies for multiple sclerosis: scientific rationale, clinical trails, and clinical practice, Current Opinion in Neurology, 20:281-285.

14. Dal Canto et al. (1977) Multiple sclerosis. Animal model: Theiler's virus infection in mice. Am. J. Path. 88:497-500.

15. De Stefano et al. (1999) “Evidence of early axonal damage in patients with multiple sclerosis”, Neurology. 52(Suppl 2):A378.

16. Dunitz, M. Multiple sclerosis therapeutics, Ed. Rudick and Goodkin. London: Taylor & Francis, 1999.

17. Durelli et al. and the Independent Comparison of Interferon (INCOMIN) Trial Study Group. (2002) “Every-other-day interferon beta-1b versus once-weekly interferon beta-1a for multiple sclerosis: results of a 2-year prospective randomised multicentre study (INCOMIN)”, Lancet. 359:1453-60.

18. EMEA Guideline on Clinical Investigation of Medicinal Products for the Treatment of Multiple Sclerosis (CPMP/EWP/561/98 Rev. 1, Nove.2006).

19. EPAR, Rebif®, Scientific Discussion.

20. Filippi et al., Glatiramer acetate reduces the proportion of MS lesions evolving into black holes, Neurol., 2001, 57:731-733.

21. Fischer et al., (1999) “The Multiple Sclerosis Functional Composite measure (MSFC): an integrated approach to MS clinical outcome assessment”, Multiple Sclerosis, 5(4):244-250.

22. Fisk et al., (1994) “Measuring the Functional Impact of Fatigue: Initial Validation of Fatigue Impact Scale”, Clin Inf Dis. 18 Suppl 1:S79-83.

23. Fisk et al., (1994) “The Impact of Fatigue on Patients with Multiple Sclerosis”, Can J Neurol Sci. 21:9-14.

24. Frenández (2007) “Combination therapy in multiple sclerosis”, Journal of the neurological sciences, 259:95-103.

25. Frohman et al., (2003) “The utility of MRI in suspected MS: report of the Therapeutics and Technology Assessment Subcommittee of the American Academy of Neurology”, Neurology. Sep. 9, 2003, 61(5):602-11.

26. Gold (2008) “Combination therapies in multiple sclerosis”, J Neurol, 255[Suppl 1]:51-60.

27. Golder W, (2007) “Magnetic resonance spectroscopy in clinical oncology”, Onkologie. 27(3): 304-9.

28. Grossman et al., (1994) Magnetization transfer: theory and clinical applications in neuroradiology”, RadioGraphics. 14:279-290.

29. Guidance for Industry. In vivo drug metabolism/drug interaction studies - study design, data analysis, and recommendations for dosing and labeling, U.S. Dept. Health and Human Svcs., FDA, Ctr. for Drug Eval. and Res., Ctr. for Biologics Eval. and Res., Clin./Pharm., November. 1999 <http ://www.fda.gov/cber/gdlns/metabol.pdf>.

30. Gurevich et al. (2010) “Laquinimod suppress antigen presentation in relapsing-remitting multiple sclerosis: in-vitro high-throughput gene expression study” (J Neuroimmunol. 2010 Apr. 15;221(1-2):87-94. Epub 2010 Mar 27.

31. Guyatt et al. (1985) “The 6-minute walk: a new measure of exercise capacity in patients with chronic heart failure”, Can Med Assoc J., 132:919-823.

32. Hafler and Weiner, MS: A CNS and systemic autoimmune disease, Immunol. Today, 1989, 10:104-107.

33. Hartung et al. (2005) “Significance of neutralizing antibodies to interferon beta during treatment of multiple sclerosis: expert opinions based on the Proceedings of an International Consensus Conference”, Eur J Neurol. 12:588-601.

34. Hauser et al. (1983) “Intensive immunosuppression in progressive multiple sclerosis”, New Engl J Med. 308:173-180.

35. Hla T, Lee M J, Ancellin N, Paik J H, Kluk M J (2001). “Lysophospholipids-receptor revelations”. Science 294 (5548): 1875-8.

36. Hohlfeld et al. (2000) “The neuroprotective effect of inflammation: implications for the therapy of multiple sclerosis”, J Neuroimmunol. 107:161-166.

37. Johnson et al., Copolymer 1 reduces relapse rate and improves disability in relapsing-remitting multiple sclerosis: results of a phase III multicenter, double-blind placebo-controlled trial. The Copolymer 1 Multiple Sclerosis Study Group, Neurol., 1995, 45:1268.

38. Kleinschmidt-DeMasters et al. (2005) New England Journal of Medicine, 353:369-379.

39. Kurtzke J F, (1983) “Rating neurologic impairment in multiple sclerosis: an expanded disability status scale (EDSS)”, Neurology 33(11):1444-1452.

40. Lampert, Autoimmune and virus-induced demyelinating diseases. A review, Am. J. Path., 1978, 91:176-208.

41. Langer-Gould et al. (2005) New England Journal of Medicine, 353:369-379.

42. Lublin FD, Reingold S C (1996) “Defining the clinical course of multiple sclerosis”, Neurol. 46:907-911.

43. Martyn, The epidemiology of multiple sclerosis, in McAlpine's Multiple Sclerosis, Matthews, B., ed., London: Churchil Livingstone, 1991, 3-40.

44. McDonald et al., Recommended diagnostic criteria for multiple sclerosis: guidelines from the International Panel on the diagnosis of multiple sclerosis. Ann. Neurol., 2001, 50:121-127.

45. Mehta et al. (1996) “Magnetization transfer magnetic resonance imaging: a clinical review”, Topics in Magnetic Resonance Imaging 8(4):214-30.

46. Miki et al. (1999) “Relapsing-Remitting Multiple Sclerosis: Longitudinal Analysis of MR Images—Lack of Correlation between Changes in T2 Lesion Volume and Clinical Findings”, Radiology. 213 :395-399.

47. Milo and Panitch (2011) “Combination therapy in multiple sclerosis”, Journal of Neuroimmunology, 231(2011):23-31.

48. Moraal et al. (2008) “Subtraction MR Images in a Multiple Sclerosis Multicenter Clinical Trial Setting”, Radiology, 250(2):506-514.

49. Multiple sclerosis: its diagnosis, symptoms, types and stages, 2003 <http://www.albany.net/˜tjc/multiple-sclerosis.html>.

50. Neuhaus et al. (2003) “Immunomodulation in multiple sclerosis: from immunosuppression to neuroprotection”, Trends Pharmacol Sci. 24:131-138.

51. Noseworthy et al. (2000) “Multiple sclerosis”, N Engl J Med. 343:938-952.

52. Olsson, Immunology of multiple sclerosis, Curr. Opin. Neurol. Neurosurg., 1992, 5:195-202.

53. Panitch et al. for the EVIDENCE (Evidence of Interferon Dose-response: European North American Comparative Efficacy) Study Group and the University of British Columbia MS/MRI Research Group. (2002) “Randomized comparative study of interferon β-1a treatment regiments in MS”, The EVIDENCE Trial. Neurology. 59:1496-1506.

54. Parkman, Graft-versus-host Disease, Ann. Rev. Med., 1991, 42: 189-197.

55. PCT International Application Publication No. WO 2007/047863, published Apr. 26, 2007, international filing date Oct. 18, 2006.

56. PCT International Application Publication No. WO 2007/146248, published Dec. 21, 2007, international filing date Jun. 12, 2007.

57. Polman et al. (2011) “Diagnostic Criteria for Multiple Sclerosis: 2010 Revisions to the McDonald Criteria”, Ann N eural, 69:292-302.

58. Polman et al., (2005) “Diagnostic criteria for multiple sclerosis: 2005 revisions to the McDonald Criteria”, Annals of Neurology, Volume 58 Issue 6, Pages 840-846.

59. Polman et al., (2005) “Treatment with laquinimod reduces development of active MRI lesions in relapsing MS”, Neurology. 64:987-991.

60. Poser et al. (1983) “New Diagnostic Criteria for Multiple Sclerosis: Guidelines for Research Protocols”, Annals of Neurology, March 1983, 13(3):227-230.

61. Rodriguez et al. (1987) Theiler's murine encephalomyelitis: a model of demyelination and persistence of virus. Crit. Rev. Immunol., 7:325.

62. Rosen Y, (2007) “The Recent advances in magnetic resonance neurospectroscopy”, Neurotherapeutics. 27(3):330-45.

63. Rudick et al. (2006) New England Journal of Medicine, 354:911-923.

64. Rudick, R. (1999) “Disease-Modifying Drugs for Relapsing-Remitting Multiple Sclerosis and Future Directions for Multiple Sclerosis Therapeutics”, Neurotherpatueics. 56:1079-1084.

65. Runström et al. (2002) “Laquinimod (ABR-215062) a candidate drug for treatment of Multiple Sclerosis inhibits the development of experimental autoimmune encephalomyelitis in IFN-β knock-out mice”, (Abstract), Medicon Valley Academy, Malmoe, Sweden.

66. Runström et al. (2006) “Inhibition of the development of chronic experimental autoimmune encephalomyelitis by laquinimod (ABR-215062) in IFN-β k.o. and wild type mice” Journal of Neuroimmunology, 173(2006):69-78.

67. Sandberg-Wollheim et al. (2005) “48-week open safety study with high-dose oral laquinimod in patients”, Mult Scler. 11:S154 (Abstract).

68. Sorenson PS. (2006) “Neutralising antibodies to interferon-β-measurement, clinical relevance, and management”, J Neurol. 253[Suppl 6]:VI/16-VI/22.

69. Teitelbaum et al., Suppression of Experimental Allergic Encephalomyelitis by a Synthetic Polypeptide, Eur. J. Immunol., 1971, 1: 242-248.

70. Teitelbaum et al., Suppression of Experimental Allergic Encephalomyelitis with Basic Polymers, Eur. J. Immunol., 1973, 3: 273-279.

71. The National MS Society (USA), The Disease Modifying Drug Brochure, Oct. 19, 2006.

72. U.S. Patent Application Publication No. 2010-0322900, published Dec. 23, 2010 (Tarcic et al.).

73. U.S. Patent Application Publication No. 2011-0027219, published Feb. 3, 2011 (Tarcic et al.).

74. U.S. Patent Application Publication No. 2011-0034508, published Feb. 10, 2011 (Liat Hayardeny).

75. U.S. Patent Application Publication No. 2011-0217295, published Sep. 8, 2011 (Haviv and Tarcic).

76. U.S. Patent Application Publication No. 2011-0218179, published Sep. 8, 2011 (Haviv and Tarcic).

77. U.S. Patent Application Publication No. 2011-0218203, published Sep. 8, 2011 (Joel Kaye et al.).

78. U.S. Patent Application Publication No. 2012-0010239, published Jan. 12, 2012 (Piryatinsky et al.).

79. U.S. Patent Application Publication No. 2012-0010238, published Jan. 12, 2012 (Fristedt).

80. U.S. Patent Application Publication No. 2012-0142730, published Jun. 7, 2012 (Tarcic et al.).

81. Vollmer et al. (2008) “Glatiramer acetate after induction therapy with mitoxantrone in relapsing multiple sclerosis” Multiple Sclerosis, 00:1-8.

82. Yang et al., (2004) “Laquinimod (ABR-215062) suppresses the development of experimental autoimmune encephalomyelitis, modulates the Th1/Th2 balance and induces the Th3 cytokine TGF-β in Lewis rats”, J. Neuroimmunol. 156:3-9.

83. Yong (2002) “Differential mechanisms of action of interferon-0 and glatiramer acetate in MS” Neurology, 59:1-7.

84. Zou et al. (2002) “Suppression of experimental autoimmune neuritis by ABR-215062 is associated with altered Th1/Th2 balance and inhibited migration of inflammatory cells into the peripheral nerve tissue”, Neuropharmacology. 42:731.

85. Clinical Trials Website, article entitled “Study of Fampridine-ER Tablets in Patients With Multiple Sclerosis”, retrieved Jul. 10, 2012, <http ://clinicaltrials govict2/show?term=fampridine&cond=multiple+sclerosis&phase=2 &ran k=7>.

86. “COPAXONE®” in Physician's Desk Reference, Thompson Reuters—Physician's Desk Reference Inc., Montvale, N.J., 2008, 3110-3113.

87. U.S. Pat. No. 3,849,550, issued Nov. 19, 1974 (Teitelbaum et al).

88. U.S. Pat. No. 5,800,808, issued Sep. 1, 1998 (Konfino et al).

89. U.S. Pat. No. 5,858,964, issued Jan. 12, 1999 (Aharoni et al).

90. U.S. Pat. No. 5,981,589, issued Nov. 9, 1999 (Konfino et al).

91. U.S. Pat. No. 6,048,898, issued Apr. 11, 2000 (Konfino et al).

92. U.S. Pat. No. 6,054,430, issued Apr. 25, 2000 (Konfino et al).

93. U.S. Pat. No. 6,077,851, issued Jun 20, 2000 (Bjork et al).

94. U.S. Pat. No. 6,214,791, issued Apr. 10, 2001 (Amon et al).

95. U.S. Pat. No. 6,342,476, issued Jan. 29, 2002 (Konfino, et al).

96. U.S. Pat. No. 6,362,161, issued Mar. 26, 2002 (Konfino et al).

97. Alejandro Horga; Xavier Montalban 06/04/2008; Expert Rev Neurother. 2008;8(5):699-714.

98. U.S. Pat. No. 7,589,208, issued Sep. 15, 2009 (Jansson et al).

99. U.S. Pat. No. 7,884,208, issued Feb. 8, 2011 (Frenkel et al.).

100. RTT News Article dated Apr. 12, 11, entitled “Teva Pharma, Active Biotech Post Positive Laquinimod Phase 3 ALLEGRO Results”.

101. Teva Press Release dated Aug. 1, 2011, entitled “Results of Phase III BRAVO Trial Reinforce Unique Profile of Laquinimod for Multiple Sclerosis Treatment”.

102. U.S. Pat. No. 7,989,473, issued Aug. 2, 2011 (Patashnik et al.).

103. U.S. Pat. No. 8,178,127, issued May 15, 2012 (Safadi et al.).

104. National MS Society Website, retrieved Jul. 10, 2012 <http://www.nationalmssociety.org/for-professionals/researchers/clinical-study-measures/t25-

105. Salama et al. (2003) Multiple Sclerosis, 9:28-31.

What is claimed is:

1. A method of treating a human patient afflicted with multiple sclerosis or presenting a clinically isolated syndrome comprising orally administering to the patient a daily dose of 0.6 mg laquinimod, and periodically administering to the patient a pharmaceutically effective amount of interferon-β, wherein the amounts when taken together is more effective to treat the human patient than when each agent is administered alone.

2. The method of claim 1, wherein the multiple sclerosis is relapsing multiple sclerosis.

3. The method of claim 2, wherein the relapsing multiple sclerosis is relapsing-remitting multiple sclerosis.

4. The method of any one of claims 1-3, wherein the amount of laquinimod and the amount of interferon-β when taken together is effective to reduce a symptom of multiple sclerosis in the human patient.

5. The method of claim 4, wherein the symptom is a MRI-monitored multiple sclerosis disease activity, relapse rate, accumulation of physical disability, frequency of relapses, frequency of clinical exacerbation, brain atrophy, risk for confirmed progression, or time to confirmed disease progression.

6. The method of claim 5, wherein the accumulation of physical disability is assessed by the time to confirmed disease progression as measured by Kurtzke Expanded Disability Status Scale (EDSS) score.

7. The method of claim 6, wherein the patient had an EDSS score of 0-5.5 prior to administration of laquinimod.

8. The method of claim 6, wherein the patient had an EDSS score of 5.5 or greater prior to administration of laquinimod.

9. The method of any one of claims 6-8, wherein confirmed disease progression is a 1 point increase of the EDSS score.

10. The method of any one of claims 6-8, wherein confirmed disease progression is a 0.5 point increase of the EDSS score.

11. The method of any one of claims 5-10, wherein time to confirmed disease progression is increased by 20-60%.

12. The method of claim 11, wherein time to confirmed disease progression is increased by at least 50%.

13. The method of any one of claims 1-12, wherein laquinimod is laquinimod sodium.

14. The method of any one of claims 1-13, wherein the interferon-β is administered via subcutaneous injection or intramuscular injection.

15. The method of claim 14, wherein the interferon-β is interferon beta-1a and is administered intramuscularly at 30 mcg, once weekly.

16. The method of claim 14, wherein the interferon-β is interferon beta-1b and is administered subcutaneously at 0.25 mg, every other day.

17. The method of claim 14, wherein the interferon-β is interferon beta-1b and is administered subcutaneously at 0.25 mg, every other day.

18. The method of claim 14, wherein the interferon-β is interferon beta-1a and is administered subcutaneously at 22-44 mcg, three times a week.

19. The method of any one of claims 1-18, wherein the administration of laquinimod substantially precedes the administration of interferon-β.

20. The method of any one of claims 1-18, wherein the administration of interferon-β substantially precedes the administration of laquinimod.

21. The method of claim 20, wherein the human patient is receiving interferon-β therapy prior to initiating laquinimod therapy.

22. The method of claim 21, where in the human patient is receiving interferon-β therapy for at least 24 weeks prior to initiating laquinimod therapy.

23. The method of claim 22, where in the human patient is receiving interferon-β therapy for at least 28 weeks prior to initiating laquinimod therapy.

24. The method of claim 23, where in the human patient is receiving interferon-β therapy for at least 48 weeks prior to initiating laquinimod therapy.

25. The method of claim 24, where in the human patient is receiving interferon-β therapy for at least 52 weeks prior to initiating laquinimod therapy.

26. The method of any one of claims 1-25, further comprising administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

27. The method of any one of claims 1-26, wherein the periodic administration of laquinimod and interferon-β continues for more than 30 days.

28. The method of claim 27, wherein the periodic administration of laquinimod and interferon-β continues for more than 42 days.

29. The method of claim 28, wherein the periodic administration of laquinimod and interferon-β continues for 6 months or more.

30. The method of any one of claims 1-29, wherein the administration of laquinimod and interferon-inhibits a symptom of relapsing multiple sclerosis by at least 30%.

31. The method claim 30, wherein the administration of laquinimod and interferon-β inhibits a symptom of relapsing multiple sclerosis by at least 50%.

32. The method of claim 31, wherein the administration of laquinimod and interferon-β inhibits a symptom of relapsing multiple sclerosis by more than 100%.

33. The method of any one of claims 32, wherein the administration of laquinimod and interferon-β inhibits a symptom of relapsing multiple sclerosis by more than 300%.

34. The method of claim 33, wherein the administration of laquinimod and interferon-β inhibits a symptom of relapsing multiple sclerosis by more than 1000%.

35. The method of any one of claims 1-34, wherein each of the amount of laquinimod when taken alone, and the amount of interferon-β when taken alone is effective to treat the human patient.

36. The method of any one of claims 1-34, wherein either the amount of laquinimod when taken alone, the amount of interferon-β when taken alone, or each such amount when taken alone is not effective to treat the human patient.

37. A method of treating a human patient afflicted with multiple sclerosis or presenting a clinically isolated syndrome comprising periodically administering to the patient an amount of laquinimod and an amount of interferon-β (IFN-β), wherein the amounts when taken together are effective to treat the human patient.

38. The method of claim 37, wherein the amount of laquinimod and the amount of IFN-β when taken together is more effective to treat the human patient than when each agent is administered alone.

39. The method of claim 37 or 38, wherein the multiple sclerosis is relapsing multiple sclerosis.

40. The method of claim 39, wherein the relapsing multiple sclerosis is relapsing-remitting multiple sclerosis.

41. The method of any one of claims 37-40, wherein the amount of laquinimod and the amount of interferon-β when taken together is effective to reduce a symptom of multiple sclerosis in the human patient.

42. The method of claim 41, wherein the symptom is a MRI-monitored multiple sclerosis disease activity, relapse rate, accumulation of physical disability, frequency of relapses, decreased time to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work.

43. The method of claim 42, wherein the amount of laquinimod and the amount of interferon-β when taken together is effective to decrease or inhibit reduction of brain volume.

44. The method of claim 43, wherein brain volume is measured by percent brain volume change (PBVC).

45. The method of claim 42, wherein the amount of laquinimod and the amount of interferon-β when taken together is effective to increase time to confirmed disease progression.

46. The method of claim 45, wherein time to confirmed disease progression is increased by 20-60%.

47. The method of claim 45, wherein time to confirmed disease progression is increased by at least 50%.

48. The method of claim 42, wherein the amount of laquinimod and the amount of interferon-β when taken together is effective to decrease abnormalities observed in whole Brain MTR histogram.

49. The method of claim 42, wherein the accumulation of physical disability is measured by Kurtzke Expanded Disability Status Scale (EDSS) score.

50. The method of claim 42, wherein the accumulation of physical disability is assessed by the time to confirmed disease progression as measured by Kurtzke Expanded Disability Status Scale (EDSS) score.

51. The method of claim 49 or 50, wherein the patient had an EDSS score of 0-5.5 prior to administration of laquinimod.

52. The method of claim 49 or 50, wherein the patient had an EDSS score of 1.5-4.5 prior to administration of laquinimod.

53. The method of claim 49 or 50, wherein the patient had an EDSS score of 5.5 or greater prior to administration of laquinimod.

54. The method of any one of claims 42-53, wherein confirmed disease progression is a 1 point increase of the EDSS score.

55. The method of any one of claims 42-53, wherein confirmed disease progression is a 0.5 point increase of the EDSS score.

56. The method of claim 42, wherein impaired mobility is assessed by the Timed-25 Foot Walk test.

57. The method of claim 42, wherein impaired mobility is assessed by the 12-Item Multiple Sclerosis Walking Scale (MSWS-12) self-report questionnaire.

58. The method of claim 42, wherein impaired mobility is assessed by the Ambulation Index (AI).

59. The method of claim 42, wherein impaired mobility is assessed by the Six-Minute Walk (6 MW) Test.

60. The method of claim 42, wherein impaired mobility is assessed by the Lower Extremity Manual Muscle Test (LEMMT) Test.

61. The method of claim 42, wherein the amount of laquinimod and the amount of interferon-β when taken together is effective to reduce cognitive impairment.

62. The method of claim 61, wherein cognitive impairment is assessed by the Symbol Digit Modalities Test (SDMT) score.

63. The method of claim 42, wherein general health status is assessed by the EuroQoL (EQ5D) questionnaire, Subject Global Impression (SGI) or Clinician Global Impression of Change (CGIC).

64. The method of claim 42, wherein functional status is measured by the patient's Short-Form General Health survey (SF-36) Subject Reported Questionnaire score.

65. The method of claim 42, wherein quality of life is assessed by SF-36, EQ5D, Subject Global Impression (SGI) or Clinician Global Impression of Change (CGIC).

66. The method of claim 64 or 65, wherein the patient's SF-36 mental component summary score (MSC) is improved.

67. The method of claim 64 or 65, wherein the patient's SF-36 physical component summary sore (PSC) is improved.

68. The method of claim 42, wherein fatigue is assessed by the EQ5D, the patient's Modified Fatigue Impact Scale (MFIS) score or the French valid versions of the Fatigue Impact Scale (EMIF-SEP) score.

69. The method of claim 42, wherein symptom severity on work is measured by the work productivity and activities impairment General Health (WPAI-GH) questionnaire.

70. The method of any one of claims 37-69, wherein laquinimod is laquinimod sodium.

71. The method of any one of claims 37-70, wherein the laquinimod is administered via oral administration.

72. The method of any one of claims 37-71, wherein the laquinimod is administered daily.

73. The method of any one of claims 37-71, wherein the laquinimod is administered more often than once daily.

74. The method of any one of claims 37-71, wherein the laquinimod is administered less often than once daily.

75. The method of any one of claims 37-74, wherein the amount laquinimod administered is less than 0.6 mg/day.

76. The method of any one of claims 37-74, wherein the amount laquinimod administered is 0.1-40.0 mg/day.

77. The method of claim 76, wherein the amount laquinimod administered is 0.1-2.5 mg/day.

78. The method of claim 77, wherein the amount laquinimod administered is 0.25-2.0 mg/day.

79. The method of claim 78, wherein the amount laquinimod administered is 0.5-1.2 mg/day.

80. The method of claim 76, wherein the amount laquinimod administered is 0.25 mg/day.

81. The method of claim 76, wherein the amount laquinimod administered is 0.3 mg/day.

82. The method of claim 76, wherein the amount laquinimod administered is 0.5 mg/day.

83. The method of claim 76, wherein the amount laquinimod administered is 0.6 mg/day.

84. The method of claim 76, wherein the amount laquinimod administered is 1.0 mg/day.

85. The method of claim 76, wherein the amount laquinimod administered is 1.2 mg/day.

86. The method of claim 76, wherein the amount laquinimod administered is 1.5 mg/day.

87. The method of claim 76, wherein the amount laquinimod administered is 2.0 mg/day.

88. The method of any one of claims 37-87, wherein a loading dose of an amount different form the intended dose is administered for a period of time at the start of the periodic administration.

89. The method of claim 88, wherein the loading dose is double the amount of the intended dose.

90. The method of claim 88 or 89, wherein the loading dose administered for two days at the start of the periodic administration.

91. The method of any one of claims 37-90, wherein the interferon-δ is administered via subcutaneous injection or intramuscular injection.

92. The method of claim 91, wherein the interferon-β is interferon beta-1a and is administered intramuscularly at 30 mcg, once weekly.

93. The method of claim 91, wherein the interferon-β is interferon beta-1b and is administered subcutaneously at 0.25 mg, every other day.

94. The method of claim 91, wherein the interferon-β is interferon beta-1b and is administered subcutaneously at 0.25 mg, every other day.

95. The method of claim 91, wherein the interferon-β is interferon beta-1a and is administered subcutaneously at 22-44 mcg, three times a week.

96. The method of any one of claims 37-95, wherein the administration of laquinimod substantially precedes the administration of interferon-β.

97. The method of any one of claims 37-95, wherein the administration of interferon-β substantially precedes the administration of laquinimod.

98. The method of any one of claim 37-95 or 97, wherein the human patient is receiving interferon-β therapy prior to initiating laquinimod therapy.

99. The method of claim 98, where in the human patient is receiving interferon-β therapy for at least 24 weeks prior to initiating laquinimod therapy.

100. The method of claim 99, where in the human patient is receiving interferon-β therapy for at least 28 weeks prior to initiating laquinimod therapy.

101. The method of claim 64, where in the human patient is receiving interferon-β therapy for at least 48 weeks prior to initiating laquinimod therapy.

102. The method of claim 100, where in the human patient is receiving interferon-β therapy for at least 52 weeks prior to initiating laquinimod therapy.

103. The method of any one of claims 1-101, further comprising administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

104. The method of any one of claims 37-102, wherein the periodic administration of laquinimod and interferon-β continues for at least 3 days.

105. The method claim 104, wherein the periodic administration of laquinimod and interferon-β continues for more than 30 days.

106. The method of claim 105, wherein the periodic administration of laquinimod and interferon-β continues for more than 42 days.

107. The method of claim 106, wherein the periodic administration of laquinimod and interferon-β continues for 8 weeks or more.

108. The method of claim 107, wherein the periodic administration of laquinimod and interferon-β continues for at least 12 weeks.

109. The method of claim 108, wherein the periodic administration of laquinimod and interferon-β continues for at least 24 weeks.

110. The method of claim 109, wherein the periodic administration of laquinimod and interferon-β continues for more than 24 weeks.

111. The method of claim 110, wherein the periodic administration of laquinimod and interferon-β continues for 6 months or more.

112. The method of any one of claims 37-111, wherein the administration of laquinimod and interferon-β inhibits a symptom of relapsing multiple sclerosis by at least 20%.

113. The method of claim 112, wherein the administration of laquinimod and interferon-β inhibits a symptom of relapsing multiple sclerosis by at least 30%.

114. The method of claim 113, wherein the administration of laquinimod and interferon-β inhibits a symptom of relapsing multiple sclerosis by at least 50%.

115. The method of claim 114, wherein the administration of laquinimod and interferon-β inhibits a symptom of relapsing multiple sclerosis by at least 70%.

116. The method of claim 115, wherein the administration of laquinimod and interferon-β inhibits a symptom of relapsing multiple sclerosis by more than 100%.

117. The method of claim 116, wherein the administration of laquinimod and interferon-β inhibits a symptom of relapsing multiple sclerosis by more than 300%.

118. The method of claim 117, wherein the administration of laquinimod and interferon-β inhibits a symptom of relapsing multiple sclerosis by more than 1000%.

119. The method of any one of claims 37-118, wherein each of the amount of laquinimod when taken alone, and the amount of interferon-β when taken alone is effective to treat the human patient.

120. The method of any one of claims 37-118, wherein either the amount of laquinimod when taken alone, the amount of interferon-β when taken alone, or each such amount when taken alone is not effective to treat the human patient.

121. A package comprising:
a) a first pharmaceutical composition comprising an amount of laquinimod and a pharmaceutically acceptable carrier;
b) a second pharmaceutical composition comprising an amount of interferon-β and a pharmaceutically acceptable carrier; and
c) instructions for use of the first and second pharmaceutical compositions together to treat a human patient afflicted with multiple sclerosis or presenting a clinically isolated syndrome.

122. The package of claim 121, wherein the first pharmaceutical composition is in liquid form.

123. The package of claim 121, wherein the first pharmaceutical composition is in solid form.

124. The package of claim 123, wherein the first pharmaceutical composition is in capsule form.

125. The package of claim 123, wherein the first pharmaceutical composition is in tablet form.

126. The package of claim 125, wherein the tablets are coated with a coating which inhibits oxygen from contacting the core.

127. The package of claim 126, wherein coating comprises a cellulosic polymer, a detackifier, a gloss enhancer, and pigment.

128. The package of anyone of claims 121-127, wherein the first pharmaceutical composition further comprises mannitol.

129. The package of anyone of claims 121-128, wherein the first pharmaceutical composition further comprises an alkalinizing agent.

130. The package of claim 129, wherein the alkalinizing agent is meglumine.

131. The package of anyone of claims 121-130, wherein the first pharmaceutical composition further comprises an oxidation reducing agent.

132. The package of anyone of claims 121-128, wherein the first pharmaceutical composition is stable and free of an alkalinizing agent or an oxidation reducing agent.

133. The package of claim 132, wherein the first pharmaceutical composition is free of an alkalinizing agent and free of an oxidation reducing agent.

134. The package of anyone of claims 121-133, wherein the first pharmaceutical composition is stable and free of disintegrant.

135. The package of anyone of claims 121-134, wherein the first pharmaceutical composition further comprises a lubricant.

136. The package of claim 135, wherein the lubricant is present in the composition as solid particles.

137. The package of claim 135 or 136, wherein the lubricant is sodium stearyl fumarate or magnesium stearate.

138. The package of anyone of claims 121-137, wherein the first pharmaceutical composition further comprises a filler.

139. The package of claim 138, wherein the filler is present in the composition as solid particles.

140. The package of claim 138 or 139, wherein the filler is lactose, lactose monohydrate, starch, isomalt, mannitol, sodium starch glycolate, sorbitol, lactose spray dried, lactose anhydrouse, or a combination thereof.

141. The package of claim 140, wherein the filler is mannitol or lactose monohydrate.

142. The package of anyone of claims 121-141, further comprising a desiccant.

143. The package of claim 142, wherein the desiccant is silica gel.

144. The package of anyone of claims 121-143, wherein the first pharmaceutical composition is stable has a moisture content of no more than 4%.

145. The package of anyone of claims 121-144, wherein laquinimod is present in the composition as solid particles.

146. The package of anyone of claims 121-145, wherein the package is a sealed packaging having a moisture permeability of not more than 15 mg/day per liter.

147. The package of claim 146, wherein the sealed package is a blister pack in which the maximum moisture permeability is no more than 0.005 mg/day.

148. The package of claim 146, wherein the sealed package is a bottle.

149. The package of claim 148, wherein the bottle is closed with a heat induction liner.

150. The package of anyone of claims 146-149, wherein the sealed package comprises an HDPE bottle.

151. The package of anyone of claims 146-150, wherein the sealed package comprises an oxygen absorbing agent.

152. The package of claim 151, wherein the oxygen absorbing agent is iron.

153. The package of any one of claims 121-152, wherein the amount of laquinimod in the first composition is less than 0.6 mg.

154. The package of any one of claims 121-152, wherein the amount of laquinimod in the first composition is 0.1-40.0 mg.

155. The package of claim 154, wherein the amount of laquinimod in the first composition is 0.1-2.5 mg.

156. The package of claim 155, wherein the amount of laquinimod in the first composition is 0.25-2.0 mg.

157. The package of claim 156, wherein the amount of laquinimod in the first composition is 0.5-1.2 mg.

158. The package of claim 154, wherein the amount of laquinimod in the first composition is 0.25 mg.

159. The package of claim 154, wherein the amount of laquinimod in the first composition is 0.3 mg.

160. The package of claim 154, wherein the amount of laquinimod in the first composition is 0.5 mg.

161. The package of claim 118, wherein the amount of laquinimod in the first composition is 0.6 mg.

162. The package of claim 154, wherein the amount of laquinimod in the first composition is 1.0 mg.

163. The package of claim 154, wherein the amount of laquinimod in the first composition is 1.2 mg.

164. The package of claim 154, wherein the amount of laquinimod in the first composition is 1.5 mg.

165. The package of claim 154, wherein the amount of laquinimod in the first composition is 2.0 mg.

166. Laquinimod for use as an add-on therapy or in combination with interferon-β in treating a human patient afflicted with multiple sclerosis or presenting a clinically isolated syndrome.

167. A pharmaceutical composition comprising an amount of laquinimod and an amount of interferon-β for use in treating a human patient afflicted with multiple sclerosis or presenting a clinically isolated syndrome, wherein the laquinimod and the interferon-β are administered simultaneously or contemporaneously.

168. A pharmaceutical composition comprising an amount of laquinimod and an amount of interferon-β.

169. The pharmaceutical composition of claim 167 or 168, in liquid form.

170. The pharmaceutical composition of claim 167 or 168, in solid form.

171. The pharmaceutical composition of claim 170, in capsule form.

172. The pharmaceutical composition of claim 170, in tablet form.

173. The pharmaceutical composition of claim 172, wherein the tablets are coated with a coating which inhibits oxygen from contacting the core.

174. The pharmaceutical composition of claim 173, wherein coating comprises a cellulosic polymer, a detackifier, a gloss enhancer, and pigment.

175. The pharmaceutical composition of anyone of claims 167-174, further comprising mannitol.

176. The pharmaceutical composition of anyone of claims 167-175, further comprising an alkalinizing agent.

177. The pharmaceutical composition of claim 176, wherein the alkalinizing agent is meglumine.

178. The pharmaceutical composition of anyone of claims 167-177, further comprising an oxidation reducing agent.

179. The pharmaceutical composition of anyone of claims 167-175, which is free of an alkalinizing agent or an oxidation reducing agent.

180. The pharmaceutical composition of claim 179, which is free of an alkalinizing agent and free of an oxidation reducing agent.

181. The pharmaceutical composition of anyone of claims 167-180, which is stable and free of disintegrant.

182. The pharmaceutical composition of anyone of claims 167-181, further comprising a lubricant.

183. The pharmaceutical composition of claim 182, wherein the lubricant is present in the composition as solid particles.

184. The pharmaceutical composition of claim 182 or 183, wherein the lubricant is sodium stearyl fumarate or magnesium stearate.

185. The pharmaceutical composition of anyone of claims 167-184, further comprising a filler.

186. The pharmaceutical composition of claim 185, wherein the filler is present in the composition as solid particles.

187. The pharmaceutical composition of claim 185 or 186, wherein the filler is lactose, lactose monohydrate, starch, isomalt, mannitol, sodium starch glycolate, sorbitol, lactose spray dried, lactose anhydrouse, or a combination thereof.

188. The pharmaceutical composition of claim 187, wherein the filler is mannitol or lactose monohydrate.

189. The pharmaceutical composition of any one of claims 167-188, wherein the amount of laquinimod in the composition is less than 0.6 mg.

190. The pharmaceutical composition of any one of claims 167-188, wherein the amount of laquinimod in the composition is 0.1-40.0 mg.

191. The pharmaceutical composition of claim 190, wherein the amount of laquinimod in the composition is 0.1-2.5 mg.

192. The pharmaceutical composition of claim 191, wherein the amount of laquinimod in the composition is 0.25-2.0 mg.

193. The pharmaceutical composition of claim 192, wherein the amount of laquinimod in the composition is 0.5-1.2 mg.

194. The pharmaceutical composition of claim 190, wherein the amount of laquinimod in the composition is 0.25 mg.

195. The pharmaceutical composition of claim 190, wherein the amount of laquinimod in the composition is 0.3 mg.

196. The pharmaceutical composition of claim 190, wherein the amount of laquinimod in the composition is 0.5 mg.

197. The pharmaceutical composition of claim 190, wherein the amount of laquinimod in the composition is 0.6 mg.

198. The pharmaceutical composition of claim 190, wherein the amount of laquinimod in the composition is 1.0 mg.

199. The pharmaceutical composition of claim 190, wherein the amount of laquinimod in the composition is 1.2 mg.

200. The pharmaceutical composition of claim 190, wherein the amount of laquinimod in the composition is 1.5 mg.

201. The pharmaceutical composition of claim 190, wherein the amount of laquinimod in the composition is 2.0 mg.

202. Use of an amount of laquinimod and an amount of interferon-β in the preparation of a combination for treating a human patient afflicted with multiple sclerosis or presenting a clinically isolated syndrome wherein the laquinimod and the interferon-β are administered simultaneously or contemporaneously.

203. A pharmaceutical composition comprising an amount of laquinimod for use in treating a subject afflicted with multiple sclerosis or presenting a clinically isolated syndrome as an add-on therapy or in combination with interferon-β by periodically administering the pharmaceutical composition and the interferon-β to the subject.

204. A pharmaceutical composition comprising an amount of interferon-β for use treating a subject afflicted with multiple sclerosis or presenting a clinically isolated syndrome as an add-on therapy or in combination with laquinimod by periodically administering the pharmaceutical composition and the laquinimod to the subject.
